(19) United States
(12) Patent Application Publication
(10) Pub. No.: US 2026/0027014 A1
(43) Pub. Date: Jan. 29, 2026

(54) DEVICES AND METHODS RELATED TO COLORIMETRIC DETECTION FOR DENTAL USES

(71) Applicant: CaviSense Inc., Boston, MA (US)

(72) Inventors: Gili NAVEH, Boston, MA (US); Michael NATAN, Boston, MA (US); Sabesh KANGALINGAM, Boston, MA (US)

(21) Appl. No.: 18/996,483

(22) PCT Filed: Jul. 21, 2023
(86) PCT No.: PCT/US2023/070772

§ 371 Date: Jan. 17, 2025

Related U.S. Application Data
(60) Provisional application No. 63/391,036, filed on Jul. 21, 2022.

(51) Int. Cl. A61K 6/25; G01N 33/52

(52) U.S. Cl. CPC A61K 6/25; G01N 33/523

(57) ABSTRACT

The disclosure is directed to compositions and methods related to colorimetric detection for dental uses (e.g., detecting the presence or absence of a dental caries, and/or cavity or cavities, or precursor thereof), including a rapid unambiguous color indicator that allows for visualization by an unassisted eye based on a localized oral pH reading.

### CROSS REFERENCE TO RELATED APPLICATIONS

This application claims priority to U.S. Provisional Application No. 63/391,036, filed on Jul. 21, 2022, the entire contents of which are incorporated herein.

### FIELD OF THE DISCLOSURE

The present disclosure relates to devices and methods directed to detecting the presence or absence of a dental caries, and/or cavity or cavities.

### BACKGROUND

Saliva is a readily accessible body fluid that may be sampled for monitoring and diagnostics purposes generally by minimally invasive techniques. Saliva is a reservoir of electrolytes, proteins, immunoglobulins, and lubricating agents with 99% water. Saliva also serves as a buffer for oral pH regulation (e.g., maintained at a pH value of about 6.6-7.1). Dental caries, and/or cavity or cavities, is the most prevalent oral disease caused by acid secreting bacteria. Salivary pH below 5.5 is indicative of the presence of active demineralization of the teeth without wishing to be bound by theory. Dental caries, and/or cavity or cavities, are caused by acidic salivary pH which results in an imbalance between the demineralization and remineralization processes of the enamel, as protons causes the decomposition of the hydroxyapatite crystals:

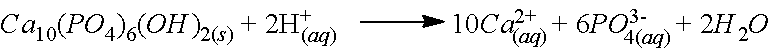

Hydroxyapatite dissolution can be reversed in a remineralization process provided a sufficient number calcium, phosphorous, or fluoride ions.

Detection of an acidic local environment below a pH threshold of 5.5 is indicative of the tipping of the salivary buffering capacity towards active demineralization of the tooth. Local detection of such imbalance thus provides an early indication of tooth demineralization before it matures into full decay.

While current detection methods, including X-ray technology, are able to detect dental caries, and/or cavity or cavities, current methods are limited to dental practices and are unable to provide information on cavity activity or provide localized monitoring. Thus, there remains a significant need for compositions and methods that improve the detection and monitoring of a dental caries, and/or cavity or cavities.

### SUMMARY

Accordingly, in various aspects, the present disclosure relates to devices and methods that detect the presence or absence of a dental caries, and/or cavity or cavities, as well as information on cavity activity using a rapid unambiguous color indicator that allows for visualization by an unassisted eye, or optionally with image analysis software.

In various aspects, disclosed herein is a device for detection of a presence or absence of a dental caries, and/or cavity or cavities, and/or cavity activity risk, and/or caries risk, or precursor thereof, comprising: (a) a support, the support being configured for grasping; (b) a substrate attached to or layered upon the support and being suitable for absorption or containing a sensing agent and detection of the sensing agent without interference; and (c) a sensing agent, the sensing agent being suitable for providing a color change which is indicative of a pH value associated with the presence or absence of a dental caries, and/or cavity or cavities, or precursor thereof. In some embodiments, the device is suitable for contacting a surface of a tooth. In some embodiments, the contacting is interproximal. In some embodiments, the support is or comprises a dental pick, toothpick, paper point, rubber tip stimulator, a gum stimulator, dental floss, gel form, tray, or test strip.

In some embodiments, the sensing agent is substantially absorbed into the substrate. In some embodiments, the sensing agent covers substantially the entire external surface of the substrate. In some embodiments the sensing agent is contained within the substrate.

In some embodiments, the substrate is or comprises a biologically compatible polymer matrix or biopolymer matrix material. In some embodiments, the sensing agent is or comprises a silk fibroin derived from a silkworm, or a spider. In some embodiments, the substrate is or comprises a naturally occurring carbonaceous material. In some embodiments, the naturally occurring carbonaceous material is activated carbon. In some embodiments, the substrate is modified to absorb the sensing agent. In some embodiments, the substrate is modified to provide an unobfuscated color signal. In some embodiments, the substrate is modified to reduce porosity relative to an unmodified form. In some embodiments, the substrate is modified to reduce porosity and increase external surface exposure to sensing agent, relative to an unmodified form.

In some embodiments, the sensing agent is or comprises a food coloring compound, or the sensing agent is or comprises a food extract compound of food coloring compound. In some embodiments, the food coloring compound or food extract compound is selected from one or more of a carotenoids, chlorophyll, anthocyanin, and turmeric, or a derivative thereof. In some embodiments, the food coloring compound or food extract compound is a beta-carotene molecule, anthocyanin, carminic acid, or a derivative thereof.

In some embodiments, the sensing agent is or comprises a food-derived agent. In some embodiments, the sensing agent is or comprises an anthocyanin or derivative thereof.

In some embodiments, the device is suitable for displaying a color change of a first color to a second color that indicates a specific pH value. In some embodiments, the device is suitable for displaying a color change of a first color to a second color that does not indicate a specific pH value. In some embodiments, the device is suitable for displaying a color change of colored to colorless, or substantially colorless, that indicates a specific pH value. In some embodiments, the device is suitable for displaying a color change of colorless, or substantially colorless, to colored that indicates a specific pH value. In some embodiments, the device is suitable for displaying a color change of colored to colorless, or substantially colorless, that does not indicate a specific pH value. In some embodiments, the device is suitable for displaying a color change of colorless to colored, or substantially colorless, that does not indicate a specific pH value. In some embodiments, the device is suitable for displaying a color change of colorless that is revealing or disappearing of a pattern that is embedded on the substrate, wherein stripes disappear and/or appear when the test is positive or negative. In some embodiments, the device is suitable for at-home detection.

In some embodiments, the device is suitable for minimally invasive detection. In some embodiments, the device detects demineralization, optionally of calcium, fluoride, and/or phosphate ions, optionally from enamel and/or dentin, optionally of enamel producing proteins and/or periodontal ligament degrading enzymes, optionally wherein the dentin and enamel producing proteins are selected from collagen and amelogenin, optionally wherein the dentin or periodontal ligament degrading enzyme is collagenase, optionally wherein the periodontal ligament degrading enzyme differentiates between deep and early stage cavities, optionally wherein the device detects demineralization of a catabolic enzyme, and optionally wherein the catabolic enzyme is a protease, sucrase, or sucrose synthase. In some embodiments, the device detects a lack or an absence of demineralization, optionally of calcium, fluoride, and/or phosphate ions, optionally from enamel and/or dentin.

In some embodiments, the device detects activity of acid-secreting bacteria. In some embodiments, the device increases the activity of acid-secreting bacteria. In some embodiments, the device detects activity of Streptococci mutans, Streptococcus sobrinus, and/or Lactobacilli spp. In some embodiments, the device detects lack of activity of acid-secreting bacteria. In some embodiments, the device detects lack of activity of Streptococci mutans, Streptococcus sobrinus, and/or Lactobacilli spp.

In some embodiments the device detects bacterial acid secretion after intentional activation of bacterial acid secretion. In some embodiments the device increases bacterial acid secretion and/or bacterial activity. In some embodiments, the device detects bacterial activity. In some embodiments, the device detects enhanced bacterial activity.

In some embodiments, the device detects a reversible dental lesion. In some embodiments, the device detects a remineralizable dental lesion. In some embodiments, the device detects the depth of the dental lesion.

In various aspects, disclosed herein is a method of detecting a presence or absence of a dental caries, and/or cavity or cavities, risk for caries, caries associated bacterial acid production, or precursor thereof, comprising: (a) contacting a surface of a tooth with the device of any one of the embodiments disclosed herein, or with the sensing agent of any one of the embodiments disclosed herein, or a support and/or substrate comprising the sensing agent of any one the embodiments disclosed herein; and (b) determining a color change in the sensing agent, the color change being indicative of a pH value that is associated with the presence or absence of tooth demineralization, dental caries, risk for caries, caries associated bacterial acid production, and/or cavity or cavities, or precursor thereof, and/or risk of developing dental caries, and/or cavity or cavities presence or activity.

In various aspects, disclosed herein is a method of detecting a presence or absence of a dental caries, and/or cavity or cavities, or precursor thereof, comprising: (a) contacting a surface of a tooth with a sensing agent, or a support and/or substrate comprising the sensing agent, the sensing agent being configured to colorimetrically detect pH of saliva, plaque, or gingival crevicular fluid; and (b) determining a color change in the sensing agent, the color change being indicative of a pH value that is associated with the presence or absence of tooth demineralization, dental caries, caries risk, caries activity level, caries associated bacterial activity, and/or cavity or cavities, or precursor thereof, and/or risk of developing dental caries, and/or cavity or cavities presence or activity.

In some embodiments, wherein the contacting is interproximal. In some embodiments, the dental caries, and/or cavity or cavities, or precursor thereof is not readily detectable with visual-tactile examination. In some embodiments, the method provides at-home detection. In some embodiments, the method provides for minimally invasive detection. In some embodiments, the method provides a signal that is visualizable by an unassisted eye. In some embodiments, the method provides a signal through a device (e.g., a phone application), and/or is based on an image analysis algorithm. In some embodiments, the method provides a signal that is visualizable by a software through taking an image of the device.

In some embodiments, the method augments or supplants a cavity detection method, optionally wherein the method augments or supplants radiographic detection, optionally bitewing radiography. In some embodiments, the method detects an active caries and/or cavity, or a lack of an active caries and/or cavity, optionally wherein the sensing agent detects the presence or absence of a dental caries, and/or cavity or cavities, for all surfaces of the teeth of a subject.

In some embodiments, the pH value is about 5.5. In some embodiments, the pH value is not about 5.5.

In some embodiments, the pH value is about 5.5 and indicative of the presence of a dental caries, and/or cavity or cavities, or precursor thereof, and/or a risk of developing dental caries.

In some embodiments, the pH value is not about 5.5 and indicative of the absence of a dental caries, and/or cavity or cavities, or precursor thereof.

In some embodiments, the method detects demineralization, optionally of calcium, fluoride, and/or phosphate ions, optionally from enamel and/or dentin, optionally of enamel producing proteins and/or periodontal ligament degrading enzymes, optionally wherein the dentin and enamel producing proteins are selected from collagen and amelogenin, optionally wherein the dentin or periodontal ligament degrading enzyme is collagenase, optionally wherein the periodontal ligament degrading enzyme differentiates between deep and early stage cavities, optionally wherein the device detects demineralization of a catabolic enzyme, and optionally wherein the catabolic enzyme is a protease, sucrase, or sucrose synthase. In some embodiments, the method detects a lack or an absence of demineralization, optionally of calcium, fluoride, and/or phosphate ions, optionally from enamel and/or dentin.

In some embodiments, the method detects activity of acid-secreting bacteria. In some embodiments, the method increases the activity of acid-secreting bacteria.

In some embodiments, the method detects activity of Streptococci mutans, Streptococcus sobrinus, and/or Lactobacilli spp. In some embodiments, the method detects lack of activity of acid-secreting bacteria. In some embodiments, the method detects lack of activity of Streptococci mutans, Streptococcus sobrinus, and/or Lactobacilli spp.

In some embodiments the method detects bacterial acid secretion after intentional activation of bacterial acid secretion. In some embodiments the method increases bacterial acid secretion and/or bacterial activity. In some embodiments, the method detects bacterial activity. In some embodiments, the method detects enhanced bacterial activity.

In some embodiments, the method detects a dental lesion. In some embodiments, the method distinguishes between active and arrested lesions. In some embodiments, the method detects an incipient dental lesion. In some embodiments, the method detects a reversible dental lesion. In some embodiments, the method detects a remineralizable dental lesion. In some embodiments, the method detects the depth of the dental lesion.

In some embodiments, the support is or comprises a dental pick, toothpick, paper point, rubber tip stimulator, a gum stimulator, dental floss, gel form, tray, or test strip.

In some embodiments, the sensing agent is substantially absorbed into the substrate. In some embodiments, the sensing agent covers substantially the entire external surface of the substrate. In some embodiments the sensing agent is contained within the substrate.

In some embodiments, the substrate is or comprises a biologically compatible polymer matrix or biopolymer matrix material. In some embodiments, the sensing agent is or comprises a silk fibroin derived from a silkworm, or a spider. In some embodiments, the substrate is or comprises a naturally occurring carbonaceous material. In some embodiments, the naturally occurring carbonaceous material is activated carbon. In some embodiments, the substrate is modified to absorb the sensing agent. In some embodiments, the substrate is modified to provide an unobfuscated color signal. In some embodiments, the substrate is modified to reduce porosity relative to an unmodified form. In some embodiments, the substrate is modified to reduce porosity and increase external surface exposure to sensing agent relative to an unmodified form.

In some embodiments, the sensing agent is or comprises a food-derived agent. In some embodiments, the sensing agent is or comprises an anthocyanin or derivative thereof.

In some embodiments, the method is suitable for displaying a color change of a first color to a second color that indicates a specific pH value. In some embodiments, the method is suitable for displaying a color change of a first color to a second color that does not indicate a specific pH value. In some embodiments, the method is suitable for displaying a color change of colored to colorless, or substantially colorless, that indicates a specific pH value. In some embodiments, the method is suitable for displaying a color change of colorless, or substantially colorless, to colored that indicates a specific pH value. In some embodiments, the method is suitable for displaying a color change of colored to colorless, or substantially colorless, that does not indicate a specific pH value. In some embodiments, the method is suitable for displaying a color change of colorless, or substantially colorless, to colored that does not indicate a specific pH value.

In various aspects, disclosed herein is a method of detecting a response to a remineralization treatment of a dental caries, and/or cavity or cavities, in a subject, comprising: (a) contacting a surface of a tooth with the device of any one of the embodiments disclosed herein, or with the sensing agent of any one of the embodiments disclosed herein, or a support and/or substrate comprising the sensing agent of any one of claims of any one of the embodiments disclosed herein; and (b) determining a color change in the sensing agent, the color change being indicative of a pH value that is associated with the presence or absence of tooth demineralization, dental caries, and/or cavity or cavities, and/or risk of developing dental caries, and/or cavity or cavities, wherein the lack of a pH value associated with the presence of a dental caries, and/or cavity or cavities, is indicative of a therapeutic response to the remineralization treatment.

In various aspects, disclosed herein is a method of detecting a response to a remineralization treatment of a dental caries, and/or cavity or cavities, in a subject, comprising: (a) contacting a surface of a tooth of the subject's with a sensing agent, or a support and/or substrate comprising the sensing agent, the sensing agent being configured to colorimetrically detect pH of saliva, plaque, or gingival crevicular fluid; and (b) determining a color change in the sensing agent, the color change being indicative of a pH value that is associated with the presence or absence of tooth demineralization, dental caries, and/or cavity or cavities, and/or risk of developing dental caries, and/or cavity or cavities, wherein the lack of a pH value associated with the presence of a dental caries, and/or cavity or cavities, is indicative of a therapeutic response to a remineralization treatment.

In some embodiments, the remineralization treatment is selected from a fluoride agent, a calcium agent, a fluoride agent, and/or a phosphate agent. In some embodiments, the fluoride agent is one or more of sodium fluoride, stannous fluoride, and acidulated phosphate fluoride. In some embodiments, the method prevents the need for a filling, crown, root canal, or extraction, optionally wherein the sensing agent detects the presence or absence of a dental caries, and/or cavity or cavities, for all surfaces of the teeth of the subject.

In various aspects, disclosed herein is a method of preparing an anthocyanin concentrate from a food and/or spice source, comprising: (a) obtaining the food and/or spice source; and (b) lyophilizing the food and/or spice source or an extract from the food and/or spice source into a powder. In some embodiments, the method further comprises: (c) optionally dissolving the powder, or a medium containing the powder; (d) optionally vacuum filtering the powder to remove any fibrous or insoluble material associated with the food and/or spice source or extract; (c) optionally lyophilizing the filtrate; (f) optionally removing the sugars from the food and/or spice source; (g) optionally repeating step (e) once; and (h) optionally freeze drying the powder to produce the sensing agent concentrate.

The details of one or more examples of the disclosure are set forth in the description below. Other features or advantages of the present disclosure will be apparent from the following drawings, detailed description of several examples, and also from the appended claims. The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### BRIEF DESCRIPTION OF THE DRAWINGS

FIG. 1A, FIG. 1B, FIG. 1C, FIG. 1D, FIG. 1E, and FIG. 1F are images showing radish (FIG. 1A), onion (FIG. 1B), black current (FIG. 1C), blueberry (FIG. 1D), red cabbage (FIG. 1E), and chokeberry (FIG. 1F) extract solutions containing anthocyanin at pH levels of 3.0, 3.5, 4.0, 4.5, 5.0, 5.5, 6.0, 6.5, 7.0, 7.5, 8.0, and 8.5.

FIG. 2A, FIG. 2B, FIG. 2C, and FIG. 2D are graphs showing the absorbance spectra of 1% solution of black current (BC) (FIG. 2A), chokeberry (CB) (FIG. 2B), wild blueberry (BB) (FIG. 2C), and 0.5% red cabbage (RC) (FIG. 2D) at different wavelengths and pH levels.

FIG. 3A, FIG. 3B, FIG. 3C, and FIG. 3D are images and graphs showing wild blueberry solutions at different concentrations. FIG. 3A is an image showing different concentrations of wild blueberry (from left to right, pH 3.3, 8.0 and 5.0; concentrations of solutions from top to bottom are 0.31%, 0.63%, 1.25%, 2.5%, 5.0%, and 10%.). FIG. 3B is a graph showing the absorbance spectra of wild blueberry at a pH level of 3.0. FIG. 3C is a graph showing the absorbance spectra of wild blueberry at a pH level of 5.0. FIG. 3D is a graph showing the absorbance spectra of wild blueberry at a pH level of 8.0.

FIG. 4 is an image showing the dip coating technique.

FIG. 5A and FIG. 5B are images showing chokeberry dyes at different pH levels. FIG. 5A shows the chokeberry dye at pH levels between 5.5 and 6.5, against a black background. FIG. 5B shows the chokeberry dye at pH levels between 5.5 and 6.5, against a white background.

FIG. 6 is an image showing the process flow of anthocyanin concentration.

FIG. 7 is an image showing structural and spectral characteristics of the major naturally occurring aglycons. The chemical structure indicates the two aromatic rings (A, B), as well as the R₁ and R₂ substitution sites.

FIG. 8 is an image showing a color change, from top (dark grey) to bottom (transparent/translucent), on a surface based on a pH level less than 5.5, and the color change visualizes an underlying substrate.

### DETAILED DESCRIPTION

The present invention is based, in part, on the discovery of a device for detection of a presence or absence of a dental caries, and/or cavity or cavities, and/or cavity activity risk, and/or caries risk, or precursor thereof, using a rapid unambiguous color indicator that allows for visualization by an unassisted eye, and methods of using the same.

### Device

In various aspects, disclosed herein is a device for detection of a presence or absence of a dental caries, and/or cavity or cavities, and/or cavity activity, and/or caries risk, or precursor thereof, comprising: (a) a support, the support being configured for grasping; (b) a substrate attached to or layered upon the support and being suitable for absorption or containing a sensing agent and detection of the sensing agent without interference; and (c) a sensing agent, the sensing agent being suitable for providing a color change which is indicative of a pH value associated with the presence or absence of a dental caries, and/or cavity or cavities, or precursor thereof. In some embodiments, the device is suitable for contacting a surface of a tooth. For example, the contacting is interproximal.

In some embodiments, the support is or comprises a dental pick, toothpick, paper point, rubber tip stimulator, a gum stimulator, dental floss, gel form, tray, or test strip.

In some embodiments, the sensing agent is substantially absorbed into the substrate. In some embodiments, the sensing agent covers substantially the entire external surface of the substrate. In some embodiments the sensing agent is contained within the substrate.

In some embodiments, the substrate is or comprises a biologically compatible polymer matrix or biopolymer matrix material. In some embodiments, the substrate is or comprises one or more of a fibroin, silk fibroin, actin, alginate, collagen, catenin, claudin, coilin, elastin, elaunin, extensin, fibrillin, keratin, lamin, laminin, tublin, viral structural protein, zein protein, polyethylene oxide, polyethylene glycol, fibronectin, polyaspartic acid, polylysine, alginate, chitosan, chitin, hyaluronic acid, pectin, polycaprolactone, polylactic acid, polyglycolic acid, polyhydroxyalkanoate, silicone, dextran, polyanhydride, gelatin, gel, lignin, cellulose, polyanilines polypyrroles, a plastic polymer, a thermoforming polymer, a 3D printed polymer, and/or optionally a gel mixed with at least one material selected from fibroin, silk fibroin, actin, alginate, collagen, catenin, claudin, coilin, elastin, elaunin, extensin, fibrillin, keratin, lamin, laminin, tublin, viral structural protein, zein protein, polyethylene oxide, polyethylene glycol, fibronectin, polyaspartic acid, polylysine, alginate, chitosan, chitin, hyaluronic acid, pectin, polycaprolactone, polylactic acid, polyglycolic acid, polyhydroxyalkanoate, silicone, dextran, polyanhydride, gelatin, gel, lignin, cellulose, polyanilines, polypyrroles, a plastic polymer, a thermoforming polymer, and a 3D printed polymer. In some embodiments, the sensing agent is or comprises a silk fibroin derived from a silkworm or a spider. In some embodiments, the substrate is or comprises a naturally occurring carbonaceous material. In some embodiments, the naturally occurring carbonaceous material is activated carbon. In some embodiments, the silkworm is Bombyx mori.

In some embodiments, the substrate is modified to absorb the sensing agent. In some embodiments, the substrate is modified to provide an unobfuscated color signal. In some embodiments, the substrate is modified to reduce porosity relative to an unmodified form. In some embodiments, the substrate is modified to reduce porosity and increase external surface exposure to sensing agent, relative to an unmodified form. In some embodiments, the substrate is modified to filter out materials and/or agents that block and/or modify the color signal. In some embodiments, the substrate is modified to filter out as blood cells.

In some embodiments, the sensing agent is or comprises a food coloring compound, or the sensing agent is or comprises a food extract compound of food coloring compound. In some embodiments, the food coloring compound or food extract compound is selected from one or more of a carotenoids, chlorophyll, anthocyanin, and turmeric, or a derivative thereof. In some embodiments, the food coloring compound or food extract compound is a beta-carotene molecule, anthocyanin, carminic acid, or a derivative thereof.

In some embodiments, the sensing agent is or comprises a food-derived agent. In some embodiments, the sensing agent is or comprises an anthocyanin or derivative thereof. In some embodiments, the anthocyanin or derivative thereof is selected from a flower, a herb, a food and/or spice source, a fermented beverage; or wherein the anthocyanin or derivative thereof is selected from Pelargonidin-3-O-glucoside (Callistephin), Cyanidin 3-O-glucoside (Chrysanthemin), Delphinidin 3-O-glucoside (Myrtillin), Peonidin-3-O-glucoside, Petunidin-3-O-glucoside, Malvidin 3-O-glucoside (Oenin), and combinations thereof.

In some embodiments, the sensing agent is or comprises an anthocyanidin or derivative thereof. In some embodiments, the anthocyanidin or derivative thereof is selected from one or more of a flower, a herb, a food and/or spice source, a fermented beverage; or wherein the anthocyanidin or derivative thereof is selected from 3,3′,4′,5,7-Pentahydroxy-flavylium chloride (cyanidin), 3,4′,5,7-Tetrahydroxy-3′-methoxyflavylium chloride (peonidin), 3,4′,5,7-Tetrahydroxy-3′,5′-dimethoxyflavylium chloride (malvidin), 3,5,7-Trihydroxy-2-(3,4,5,trihydroxyphenyl)-1-benzopyrylium chloride (delphinidin), 3,3′4′,5,7-Pentahydroxy-5′-methoxyflavylium chloride (petunidin), 3,5,7-Trihydroxy-2-(4-hydroxyphenyl)-1-benzopyrilium chloride (pelargonidin), and combinations thereof.

In some embodiments, the sensing agent is or comprises a curcumin, or derivative thereof. In some embodiments, the sensing agent is or comprises a porhyrin, or derivative thereof.

In some embodiments, the sensing agent is solubilized in a solution and present in the substrate at a concentration of at least 0.1%, at least 0.2%, at least 0.3%, at least 0.4%, at least 0.5%, at least 0.6%, at least 0.7%, at least 0.8%, at least 0.9%, at least 1.0%, at least 1.25%, at least 1.50%, at least 1.75%, at least 2.0%, at least 2.25%, at least 2.50%, at least 5.0%, at least 10.0%, at least 11.0%, at least 12.0%, at least 13.0%, at least 14.0%, at least 15.0% at least 20.0%, at least 25.0%, at least 30.0%, at least 35.0%, at least 40.0%, at least 45.0%, or at least 50.0%.

In some embodiments, the sensing agent is present in the substrate at a concentration from at least 0.1% to at least 10%. In some embodiments, the sensing agent is present in the substrate at a concentration from at least 0.3% to at least 0.75%. In some embodiments, the sensing agent is present in the substrate at a concentration from at least 1.0% to at least 5.0%. In some embodiments, the sensing agent is present in the substrate at a concentration from at least 5.0% to at least 10.0%. In some embodiments, the sensing agent is present in the substrate at a concentration from at least 10.0% to at least 50.0%.

In some embodiments, the sensing agent is solubilized in a sugar, a carbohydrate, or a polyethylene glycol (“PEG”)-based material.

In some embodiments, the sensing agent is solubilized in methanol, ethanol, sucrose, water mixtures, or water.

In some embodiments, the sensing agent changes in hardness or consistency during any one of the embodiments disclosed herein.

In some embodiments, the solution is devoid of sugar. In some embodiments, the content of the sensing agent in the solution devoid of sugar is at a concentration of at least 0.1%, at least 0.2%, at least 0.3%, at least 0.4%, at least 0.5%, at least 0.6%, at least 0.7%, at least 0.8%, at least 0.9%, at least 1.0%, at least 1.25%, at least 1.50%, at least 1.75%, at least 2.0%, at least 2.25%, at least 2.50%, at least 5.0%, at least 10.0%, at least 11.0%, at least 12.0%, at least 13.0%, at least 14.0%, or at least 15.0%. In some embodiments, the content of the sensing agent in the solution devoid of sugar is at a concentration of at least 0.1% to 12%.

In some embodiments, the sensing agent is extracted from one or more food and/or spice sources and concentrated. For example, the one or more food and/or spice sources in some embodiments is a fruit, a vegetable, a liquid, or a spice, or a derivative thereof. In some embodiments, the one or more fruit or vegetable is selected from red cabbage, black currant, chokeberry, onion, radish, peaches, and wild blueberries. In some embodiments, the sensing agent is extracted from one or more food and/or spice sources by filtering and concentrating a lyophilized powder of the one or more food and/or spice sources, optionally wherein the device detects the presence or absence of a dental caries, and/or cavity or cavities, for all surfaces of the teeth of a subject.

In some embodiments, the device is suitable for displaying a color change of a first color to a second color that indicates a specific pH value.

In some embodiments, the device is suitable for displaying a color change of a first color to a second color that does not indicate a specific pH value.

In some embodiments, the first color is a primary color. In some embodiments, the first color is red, yellow, or blue.

In some embodiments, the first color is a secondary color. In some embodiments, the first color is orange, green, or violet.

In some embodiments, the first color is a tertiary color. In some embodiments, the first color is red-orange, yellow-orange, yellow-green, blue-green, blue-violet, or red-violet.

In some embodiments, the second color is a primary color. In some embodiments, the second color is red, yellow, or blue

In some embodiments, the second color is a secondary color. In some embodiments, the second color is orange, green, or violet.

In some embodiments, the second color is a tertiary color. In some embodiments, the second color is red-orange, yellow-orange, yellow-green, blue-green, blue-violet, or red-violet.

In some embodiments, the device is suitable for displaying a color change of colored to colorless, or substantially colorless, that indicates a specific pH value. In some embodiments, the device is suitable for displaying a color change of colorless or substantially colorless, to colored that indicates a specific pH value.

In some embodiments, the device is suitable for displaying a color change of colored to colorless, or substantially colorless, that does not indicate a specific pH value. In some embodiments, the device is suitable for displaying a color change of colorless, or substantially colorless, to colored that does not indicate a specific pH value.

In some embodiments, the sensing agent has a high absorbance. In some embodiments, the sensing agent has a high absorbance and is applied to the substrate in a monolayer or bilayer. In some embodiments, the sensing agent has a high absorbance and is applied to the substrate in solution, or on a surface.

In some embodiments, the sensing agent has a low absorbance. In some embodiments, the sensing agent has a low absorbance and is applied to the substrate in a multilayer, optionally about 3, or about 4, or about 5, or about 6, or about 7, or about 8, or about 9, or about 10 layers.

In some embodiments, the sensing agent has a low absorbance and is applied to the substrate in solution, or on a surface.

In some embodiments, the device is suitable for detecting color change by an unassisted eye when placed over a visualization surface, the visualization surface being black, reflective, matted, semi-reflective, or a color which is distinct from the color which is indicative of the pH value.

In some embodiments, the visualization surface is a primary color. In some embodiments, the visualization surface is red, yellow, or blue. In some embodiments, the visualization surface is a secondary color. In some embodiments, the visualization surface is orange, green, or violet. In some embodiments, the visualization surface is a tertiary color. In some embodiments, the visualization surface is red-orange, yellow-orange, yellow-green, blue-green, blue-violet, or red-violet.

In some embodiments, the device is suitable for detecting pH in saliva. In some embodiments, the device is suitable for detecting pH in gingival crevicular fluid. In some embodiments, the dental caries, and/or cavity or cavities, or precursor, thereof is not readily detectable with visual-tactile examination.

In some embodiments, the device is suitable for at-home detection. In some embodiments, the device is suitable for detection by an end user. In some embodiments, the device is suitable for minimally invasive detection. In some embodiments, the device provides a signal that is visualizable by an unassisted eye.

In some embodiments, the device supplants a cavity detection method, optionally wherein the device augments or supplants radiographic detection, optionally bitewing radiography, periapical radiography, panoramic radiography, or cone beam computed tomography (“CBCT”).

In some embodiments, the device detects an active caries and/or cavity, or a lack of an active caries and/or cavity. In some embodiments, the device detects active caries and/or cavity, or a lack of an active caries and/or cavity by visual-tactile. In some embodiments, the device detects active caries and/or cavity, or a lack of an active caries and/or cavity by

In some embodiments, the pH value is about 5.5. In some embodiments, the pH value is not about 5.5. In some embodiments, the pH value is about 5.5 and indicative of the presence of a dental caries, and/or cavity or cavities, or precursor thereof, and/or a risk of developing dental caries. In some embodiments, the pH value is not about 5.5 and indicative of the absence of a dental caries, and/or cavity or cavities, or precursor thereof.

In some embodiments, the device detects a pH value less than 5.5, but the output on the device does not indicate the corresponding pH value.

In some embodiments, the method detects a pH value less than 5.5, but the output on the device does not indicate the corresponding pH value.

In some embodiments, the device detects demineralization, optionally of calcium fluoride, and/or phosphate ions, optionally from enamel and/or dentin, optionally of enamel producing proteins and/or periodontal ligament degrading enzymes, optionally wherein the dentin and enamel producing proteins are selected from collagen and amelogenin, optionally wherein the dentin or periodontal ligament degrading enzyme is collagenase, optionally wherein the periodontal ligament degrading enzyme differentiates between deep and early stage cavities, optionally wherein the device detects demineralization of a catabolic enzyme, and optionally wherein the catabolic enzyme is a protease, sucrase, or sucrose synthase. In some embodiments, the device detects a lack or an absence of demineralization, optionally of calcium, fluoride, and/or phosphate ions, optionally from enamel and/or dentin.

In some embodiments, the device detects activity of acid-secreting bacteria. In some embodiments, the device increases the activity of acid-secreting bacteria. In some embodiments, the device detects activity of Streptococci mutans, Streptococcus sobrinus, and/or Lactobacilli spp. In some embodiments, the device detects lack of activity of acid-secreting bacteria. In some embodiments, the device detects lack of activity of Streptococci mutans, Streptococcus sobrinus, and/or Lactobacilli spp.

In some embodiments the device detects bacterial acid secretion after intentional activation of bacterial acid secretion. In some embodiments the device increases bacterial acid secretion and/or bacterial activity. In some embodiments, the device detects bacterial activity. In some embodiments, the device detects enhanced bacterial activity.

In some embodiments, the device detects a dental lesion. In some embodiments, the device distinguishes between active and arrested lesions. In some embodiments, the device detects an incipient dental lesion. In some embodiments, the device detects a reversible dental lesion. In some embodiments, the device detects the depth of the dental lesion. In some embodiments, the device detects a remineralizable dental lesion.

### Methods of Use

In various embodiments, disclosed herein is a method of detecting a presence or absence of a dental caries, and/or cavity or cavities, risk for caries, or caries associated with bacterial acid production, or precursor thereof, comprising: (a) contacting a surface of a tooth with a device of any one of the preceding embodiments, or with the sensing agent of any one of the preceding embodiments, or a support and/or substrate comprising the sensing agent of any one of the preceding embodiments; and (b) determining a color change in the sensing agent, the color change being indicative of a pH value that is associated with the presence or absence of tooth demineralization, dental caries, risk for caries, caries associated bacterial acid production, and/or cavity or cavities, or precursor thereof, and/or risk of developing dental caries, and/or cavity or cavities presence or activity.

In various embodiments, disclosed herein is a method of detecting a presence or absence of a dental caries, and/or cavity or cavities, risk for caries, caries associated bacterial acid production, or precursor thereof, comprising: (a) contacting a surface of a tooth with a sensing agent, or a support and/or substrate comprising the sensing agent, the sensing agent being configured to colorimetrically detect pH of saliva, plaque, or gingival crevicular fluid; and (b) determining a color change in the sensing agent, the color change being indicative of a pH value that is associated with the presence or absence of tooth demineralization, dental caries, caries risk, caries activity level, caries associated bacterial activity, and/or cavity or cavities, or precursor thereof, and/or risk of developing dental caries, and/or cavity or cavities presence or activity.

In various embodiments, the methods disclosed herein determine a color change in the sensing agent the color change being indicative of a pH value that is associated with a risk of developing dental caries, and/or cavity or cavities in a subject. In embodiments, when the pH value is about 5.5, it is indicative of the presence of a dental caries, and/or cavity or cavities, or precursor thereof, and/or a risk of developing dental caries. In embodiments, a subject at risk of developing dental caries is predisposed to the dental caries (e.g., has a personal or family history, or have a mutation in a gene that reduces enamel or otherwise causes dental caries), or show early signs or symptoms of dental caries and/or cavity or cavities. In embodiments, a subject at risk of developing dental caries can also be at risk due to environmental factors (e.g., poor oral hygiene, a diet high in sugar, other factors that reduce enamel, limited access to dental care, or absence of fluoridated water). Visual inspection, fluorescence imaging, X-rays, and/or medical/dental history can reveal a subject with or at risk of dental caries and/or cavity or cavities. The surface of the tooth may be soft when probed with a sharp instrument. Saliva or a dental plaque sample from the subject can be obtained to determine the presence or absence of a particular bacteria, for example Streptococcus mutans, in order to determine the risk of developing dental caries and/or cavity or cavities. Also, without wishing to be bound by theory, a subject currently with dental caries and/or cavity or cavities in some embodiments has one or more than one symptom and may have been previously diagnosed with the dental caries and/or cavity or cavities.

In embodiments, characteristics that place a subject at high risk of developing dental caries, and/or cavity or cavities include: sugary foods or drinks, dry mouth (Xerostomia; ages >6 years) or visually inadequate salivary flow either physiological, medicine related or due to radiation or disease, clinically detectable early, shallow, or microcavitations (moderate decay), and clinically detectable late or deep cavitations (extensive decay).

In some embodiments, the contacting is interproximal.

In some embodiments, the dental caries, and/or cavity or cavities, or precursor thereof, is not readily detectable with visual-tactile examination.

In some embodiments, the method provides at-home detection. In some embodiments, the method provides for minimally invasive detection. In some embodiments, the method provides a signal that is visualizable by an unassisted eye. In some embodiments, the method provides a signal through a device (e.g., a phone application), and/or is based on an image analysis algorithm. In some embodiments, the method provides a signal that is visualizable by a software through taking an image of the device.

In some embodiments, the method supplants a cavity detection method, optionally wherein the method augments or supplants radiographic detection, optionally bitewing radiography or other methods such as periapical radiography, panoramic radiography, or cone beam computed tomography (“CBCT”), Quantitative Light-induced Fluorescence (QLF), Fibre-optic Transillumination (FOTI), Electrical Conductance (EC), and fluorescence or dyed particles/nanoparticles.

In some embodiments, the method detects an active caries and/or cavity, or a lack of an active caries and/or cavity, optionally wherein the sensing agent detects the presence or absence of a dental caries, and/or cavity or cavities, for all surfaces of the teeth of a subject.

In some embodiments, the pH value is about 5.5. In some embodiments, the pH value is not about 5.5. In some embodiments, the pH value is about 5.5 and indicative of the presence of a dental caries, and/or cavity or cavities, or precursor thereof, and/or a risk of developing dental caries. In some embodiments, the pH value is not about 5.5 and indicative of the absence of a dental caries, and/or cavity or cavities, or precursor thereof.

In some embodiments, the method detects demineralization, optionally of calcium, fluoride, and/or phosphate ions, optionally from enamel and/or dentin, optionally of enamel producing proteins and/or periodontal ligament degrading enzymes, optionally wherein the dentin and enamel producing proteins are selected from collagen and amelogenin, optionally wherein the dentin or periodontal ligament degrading enzyme is collagenase, optionally wherein the periodontal ligament degrading enzyme differentiates between deep and early stage cavities, optionally wherein the device detects demineralization of a catabolic enzyme, and optionally wherein the catabolic enzyme is a protease, sucrase, or sucrose synthase. In some embodiments, the method detects a lack or an absence of demineralization, optionally of calcium, fluoride, and/or phosphate ions, optionally from enamel and/or dentin.

In some embodiments, the method detects activity of acid-secreting bacteria. In some embodiments, the method increases the activity of acid-secreting bacteria. In some embodiments, the method detects activity of Streptococci mutans, Streptococcus sobrinus, and/or Lactobacilli spp. In some embodiments, the method detects lack of activity of acid-secreting bacteria. In some embodiments, the method detects lack of activity of Streptococci mutans, Streptococcus sobrinus, and/or Lactobacilli spp.

In some embodiments the method detects bacterial acid secretion after intentional activation of bacterial acid secretion. In some embodiments the method increases bacterial acid secretion and/or bacterial activity. In some embodiments, the method detects bacterial activity. In some embodiments, the method detects enhanced bacterial activity.

In some embodiments, the method detects a dental lesion. In some embodiments, the method distinguishes between active and arrested lesions. In some embodiments, the method detects an incipient dental lesion. In some embodiments, the method detects a reversible dental lesion. In some embodiments, the method detects the depth of the dental lesion. In some embodiments, the method detects a remineralizable dental lesion.

In some embodiments, the support is or comprises a dental pick, toothpick, paper point, rubber tip stimulator, a gum stimulator, dental floss, gel form, tray, or test strip.

In some embodiments, the sensing agent is substantially absorbed into the substrate. In some embodiments, the sensing agent covers substantially the entire external surface of the substrate. In some embodiments the sensing agent is contained within the substrate.

In some embodiments, the substrate is or comprises a biologically compatible polymer matrix or biopolymer matrix material. In some embodiments, the substrate is or comprises one or more of a fibroin, silk fibroin, actin, alginate, collagen, catenin, claudin, coilin, elastin, elaunin, extensin, fibrillin, keratin, lamin, laminin, tublin, viral structural protein, zein protein, polyethylene oxide, polyethylene glycol, fibronectin, polyaspartic acid, polylysine, alginate, chitosan, chitin, hyaluronic acid, pectin, polycaprolactone, polylactic acid, polyglycolic acid, polyhydroxyalkanoate, silicone, dextran, polyanhydride, gelatin, lignin, cellulose, polyanilines polypyrroles, a plastic polymer, a thermoforming polymer, a 3D printed polymer, and/or optionally a gel mixed with at least one material selected from fibroin, silk fibroin, actin, alginate, collagen, catenin, claudin, coilin, elastin, elaunin, extensin, fibrillin, keratin, lamin, laminin, tublin, viral structural protein, zein protein, polyethylene oxide, polyethylene glycol, fibronectin, polyaspartic acid, polylysine, alginate, chitosan, chitin, hyaluronic acid, pectin, polycaprolactone, polylactic acid, polyglycolic acid, polyhydroxyalkanoate, silicone, dextran, polyanhydride, gelatin, gel, lignin, cellulose, polyanilines, polypyrroles, a plastic polymer, a thermoforming polymer, and a 3D printed polymer. In some embodiments, the sensing agent is or comprises a silk fibroin derived from a silkworm, or from a spider. In some embodiments, the silkworm is Bombyx mori.

In some embodiments, the substrate is modified to absorb the sensing agent. In some embodiments, the substrate is modified to provide an unobfuscated color signal. In some embodiments, the substrate is modified to reduce porosity relative to an unmodified form. In some embodiments, the substrate is modified to reduce porosity and increase external surface exposure to sensing agent relative to an unmodified form.

In some embodiments, the sensing agent is or comprises a food-derived agent. In some embodiments, the sensing agent is or comprises an anthocyanin or derivative thereof. In some embodiments, the anthocyanin or derivative thereof is selected from a flower, a herb, a food and/or spice source, a fermented beverage; or wherein the anthocyanin or derivative thereof is selected from Pelargonidin-3-O-glucoside (Callistephin), Cyanidin 3-O-glucoside (Chrysanthemin), Delphinidin 3-O-glucoside (Myrtillin), Peonidin-3-O-glucoside, Petunidin-3-O-glucoside, Malvidin 3-O-glucoside (Oenin), and combinations thereof.

In some embodiments, the sensing agent is or comprises an anthocyanidin or derivative thereof. In some embodiments, the anthocyanidin or derivative thereof is selected from one or more of a flower, a herb, a food and/or spice source, a fermented beverage; or wherein the anthocyanidin or derivative thereof is selected from 3,3′,4′,5,7-Pentahydroxy-flavylium chloride (cyanidin), 3,4′,5,7-Tetrahydroxy-3′-methoxyflavylium chloride (peonidin), 3,4′,5,7-Tetrahydroxy-3′,5′-dimethoxyflavylium chloride (malvidin), 3,5,7-Trihydroxy-2-(3,4,5,trihydroxyphenyl)-1-benzopyrylium chloride (delphinidin), 3,3′4′,5,7-Pentahydroxy-5′-methoxyflavylium chloride (petunidin), 3,5,7-Trihydroxy-2-(4-hydroxyphenyl)-1-benzopyrilium chloride (pelargonidin), and combinations thereof.

In some embodiments, the sensing agent is or comprises a curcumin, or derivative thereof. In some embodiments, the sensing agent is or comprises a porhyrin, or derivative thereof.

In some embodiments, the sensing agent is solubilized in a solution and present in the substrate at a concentration of at least 0.1%, at least 0.2%, at least 0.3%, at least 0.4%, at least 0.5%, at least 0.6%, at least 0.7%, at least 0.8%, at least 0.9%, at least 1.0%, at least 1.25%, at least 1.50%, at least 1.75%, at least 2.0%, at least 2.25%, at least 2.50%, at least 5.0%, at least 10.0%, at least 11.0%, at least 12.0%, at least 13.0%, at least 14.0%, at least 15.0% at least 20.0%, at least 25.0%, at least 30.0%, at least 35.0%, at least 40.0%, at least 45.0%, or at least 50.0%.

In some embodiments, the sensing agent is present in the substrate at a concentration from at least 0.1% to at least 10%. In some embodiments, the sensing agent is present in the substrate at a concentration from at least 0.3% to at least 0.75%. In some embodiments, the sensing agent is present in the substrate at a concentration from at least 1.0% to at least 5.0%. In some embodiments, the sensing agent is present in the substrate at a concentration from at least 5.0% to at least 10.0%. In some embodiments, the sensing agent is present in the substrate at a concentration from at least 10.0% to at least 50.0%.

In some embodiments, the sensing agent is solubilized in a sugar, a carbohydrate, or a PEG-based material.

In some embodiments, the sensing agent is solubilized in methanol, ethanol, sucrose, water mixtures, or water.

In some embodiments, the solution is devoid of sugar. In some embodiments, the content of the sensing agent in the solution devoid of sugar is at a concentration of at least 0.1%, at least 0.2%, at least 0.3%, at least 0.4%, at least 0.5%, at least 0.6%, at least 0.7%, at least 0.8%, at least 0.9%, at least 1.0%, at least 1.25%, at least 1.50%, at least 1.75%, at least 2.0%, at least 2.25%, at least 2.50%, at least 5.0%, at least 10.0%, at least 11.0%, at least 12.0%, at least 13.0%, at least 14.0%, or at least 15.0%.

In some embodiments, content of the sensing agent in the solution devoid of sugar is at a concentration of at least 0.1% to 12%.

In some embodiments, the sensing agent is extracted from one or more food and/or spice sources and concentrated. In some embodiments, the sensing agent is extracted from one or more food and/or spice sources by filtering and concentrating a lyophilized powder of the one or more food and/or spice sources, optionally wherein the device detects the presence or absence of a dental caries, and/or cavity or cavities, for all surfaces of the teeth of a subject.

In some embodiments, the method is suitable for displaying a color change of a first color to a second color that indicates a specific pH value. In some embodiments, the method is suitable for displaying a color change of a first color to a second color that does not indicate a specific pH value.

In some embodiments, the first color is a primary color. In some embodiments, the first color is red, yellow, or blue.

In some embodiments, the first color is a secondary color. In some embodiments, the first color is orange, green, or violet.

In some embodiments, the first color is a tertiary color. In some embodiments, the first color is red-orange, yellow-orange, yellow-green, blue-green, blue-violet, or red-violet.

In some embodiments, the second color is a primary color. In some embodiments, the second color is red, yellow, or blue.

In some embodiments, the second color is a secondary color. In some embodiments, the second color is orange, green, or violet.

In some embodiments, the second color is a tertiary color. In some embodiments, the second color is red-orange, yellow-orange, yellow-green, blue-green, blue-violet, or red-violet.

In some embodiments, the method is suitable for displaying a color change of colored to colorless, or substantially colorless, that indicates a specific pH value. In some embodiments, the method is suitable for displaying a color change of colorless, or substantially colorless, to colored that indicates a specific pH value.

In some embodiments, the method is suitable for displaying a color change of colored to colorless, or substantially colorless, that does not indicate a specific pH value. In some embodiments, the method is suitable for displaying a color change of colorless, or substantially colorless, to colored that does not indicate a specific pH value.

In some embodiments, the sensing agent has a high absorbance. In some embodiments, the sensing agent has a high absorbance and is applied to the substrate in a monolayer or bilayer. In some embodiments, the sensing agent has a high absorbance and is applied to the substrate in solution, or on a surface.

In some embodiments, the sensing agent has a low absorbance. In some embodiments, the sensing agent has a low absorbance and is applied to the substrate in a multilayer, optionally about 3, or about 4, or about 5, or about 6, or about 7, or about 8, or about 9, or about 10 layers.

In some embodiments, the method is suitable for detecting color change by an unassisted eye when placed over a visualization surface, the visualization surface being black, reflective, matted, semi-reflective, or a color which is distinct from the starting or target detection color.

In some embodiments, the visualization surface is a primary color. In some embodiments, the visualization surface is red, yellow, or blue.

In some embodiments, the visualization surface is a secondary color. In some embodiments, the visualization surface is orange, green, or violet.

In some embodiments, the visualization surface is a tertiary color. In some embodiments, the visualization surface is red-orange, yellow-orange, yellow-green, blue-green, blue-violet, or red-violet.

In some embodiments, the method is suitable for detecting pH in saliva.

In some embodiments, the method is suitable for detecting pH in gingival crevicular fluid.

In various aspects, disclosed herein is a method of detecting a response to a remineralization treatment of a dental caries, and/or cavity or cavities, in a subject, comprising: (a) contacting a surface of a tooth with the device of any one of the embodiments disclosed herein, or with the sensing agent of any one of the preceding embodiments, or a support and/or substrate comprising the sensing agent of any one of the preceding embodiments; and (b) determining a color change in the sensing agent, the color change being indicative of a pH value that is associated with the presence or absence of tooth demineralization, dental caries, and/or cavity or cavities, and/or risk of developing dental caries, and/or cavity or cavities, wherein the lack of a pH value associated with the presence of a dental caries, and/or cavity or cavities, is indicative of a therapeutic response to a remineralization treatment, or to an improved oral hygiene.

In various aspects, disclosed herein is a method of detecting a response to a remineralization treatment of a dental caries, and/or cavity or cavities, in a subject, comprising: (a) contacting a surface of a tooth of the subject's with a sensing agent, or a support and/or substrate comprising the sensing agent, the sensing agent being configured to colorimetrically detect pH of saliva, plaque, or gingival crevicular fluid; and (b) determining a color change in the sensing agent, the color change being indicative of a pH value that is associated with the presence or absence of tooth demineralization, dental caries, and/or cavity or cavities, and/or risk of developing dental caries, and/or cavity or cavities, wherein the lack of a pH value associated with the presence of a dental caries, and/or cavity or cavities, is indicative of a therapeutic response to a remineralization treatment, or to an improved oral hygiene.

In some embodiments, the remineralization treatment is selected from a fluoride agent, a calcium agent, and/or a phosphate agent. In some embodiments, the fluoride agent is one or more of sodium fluoride, stannous fluoride, and acidulated phosphate fluoride. In some embodiments, the methods prevents the need for a filling, crown, root canal, or extraction.

In various aspects, disclosed herein is a method of a making a sensing agent. In some embodiments a sensing agent can be made from a food and/or spice source, comprising: (a) obtaining the food and/or spice source; and (b) lyophilizing the food and/or spice source or an extract from the food and/or spice source into a powder. In some embodiments, the method further comprises: (c) optionally dissolving the powder, or a medium containing the powder; (d) optionally vacuum filtering the powder to remove any fibrous or insoluble material associated with the food and/or spice source or extract; (e) optionally lyophilizing the filtrate; (f) optionally removing the sugars from the food and/or spice source; (g) optionally repeating step (e) once; and (h) optionally freeze drying the powder to produce the sensing agent concentrate.

In various aspects, disclosed herein is a method of preparing an anthocyanin, a carotene, or a curcumin concentrate from a food and/or spice source, comprising: (a) obtaining the food and/or spice source; and (b) lyophilizing the food and/or spice source or an extract from the food and/or spice source into a powder. In some embodiments, the method further comprises: (c) optionally dissolving the powder, or a medium containing the powder; (d) optionally vacuum filtering the powder to remove any fibrous or insoluble material associated with the food and/or spice source or extract; (e) optionally lyophilizing the filtrate; (f) optionally removing the sugars from the food and/or spice source; (g) optionally repeating step (e) once; and (h) optionally freeze drying the powder to produce the sensing agent concentrate.

In some embodiments, the food and/or spice source is a fruit- or vegetable or spice-derived material. In some embodiments, the powder is dissolved in water at about a 20% weight for weight proportion. In some embodiments, the lyophilizing is at a pressure of 0.2 millibars. In some embodiments, the sugars from the food and/or spice source are removed by dissolving the filtrate in methanol, sucrose, or water. In some embodiments, the sugars are dissolved in methanol at about a 20% weight for weight proportion. In some embodiments, the prepared anthocyanin concentrate is dissolved in water at about a 50% weight for weight proportion. In some embodiments, the prepared anthocyanin concentrate is for dip coating.

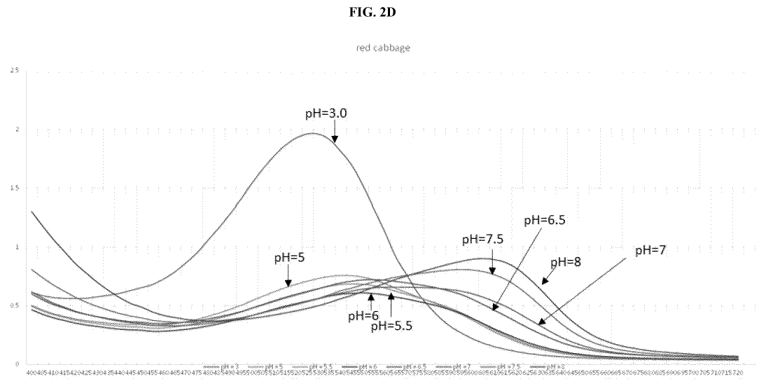

In some embodiments, a color change is visualized as shown in FIG. 8

In some embodiments, a color change occurs on a surface from yellow to transparent/translucent when the pH level is less than 5.5. In some embodiments, the color change visualizes an underlying substrate. In some embodiments, when the pH is less than 5.5, the colored substrate and colored dye become transparent/translucent, allowing for visualization of the substrate. In some embodiments, the reaction also reverses with colorless dye that becomes colored and blocks the substrate color at pH<5.5. In some embodiments, in some embodiments there is a “one-color-change” implementation. In some embodiments, the user sees one color, then another color at pH 5.5, but one of the colors is the substrate. In some embodiments, the color change is in the surface from colored to colorless, or substantially colorless, thereby exposing the substrates blueness in this example. In some embodiments, the visualization of the signal is the reflectivity of the surface. The reflectivity of the surface has two components: the reflectivity of the sensing agent, and the reflectivity of the substrate. In some embodiments, the reflectivity of the sensing agent, and the reflectivity of the substrate can be visualized independently or in combination.

### Dental Caries, and or Cavity or Cavities,

Dental caries, and/or cavity or cavities, at early stages are typically incipient lesions, can be remineralized and thus are fully reversible. As used herein, “dental caries” is interchangeable with “cavity” and/or “cavities.” Interventions such as high fluoride, calcium and phosphor-based pastes already exist in the market. These treatments can be used in the dental chair or at home. However to date, there is a limited capability for measuring localized salivary pH to assess and monitor the efficiency of these aforementioned treatment modalities, specifically from the home setting. As disclosed herein, localized salivary pH diagnostics that can be used both in the dentist chair and at home provide better diagnostics and treatment monitoring for both the patient and dentist. For best utility as a diagnostic tool, a device for such purposes should allow for localized, personalized, and continuous (daily or weekly) sampling of saliva with a low volume of saliva (in μL range). The result would be rapidly visible to the user (e.g., within seconds), and conclusively with high precision and accuracy. Regular and repeated use of the device would allow for tracking and monitoring over time.

Disclosed herein is an inexpensive, rapid, and single use colorimetric sensing device, that provides unambiguous detection of local pH, within pH range of 5-6, in localized area. The sensing device disclosed herein is colorimetric and produced from edible biomaterials functionalized onto oral devices, such as toothpick like probe, moldable material, or a tray to rapidly and unambiguously determine local pH by contacting the targeted tissue in a binary manner. As disclosed herein, methods of developing the device include: (1) selecting the choice of dye to yield the sharpest possible yes/no (binary) transition for an unassisted eye at pH 5.5, (2) selection of dye concentration and dye layering/spacing to achieve the maximum visible color change given fixed volume of body fluid such, as saliva or gingival crevicular fluid; (3) using a black background to visualize the color changes under different lighting conditions; and (4) adjusting the starting pH to facilitate ready observation of color change.

As disclosed herein, local pH is found to be less than 5.5 in an active demineralization process. A sensor color coated device that changes color upon detection of pH less than 5.5 readily visible by unassisted eye in the dental office or at home is described herein. Additionally, the color changing sensor is obtained from edible materials.

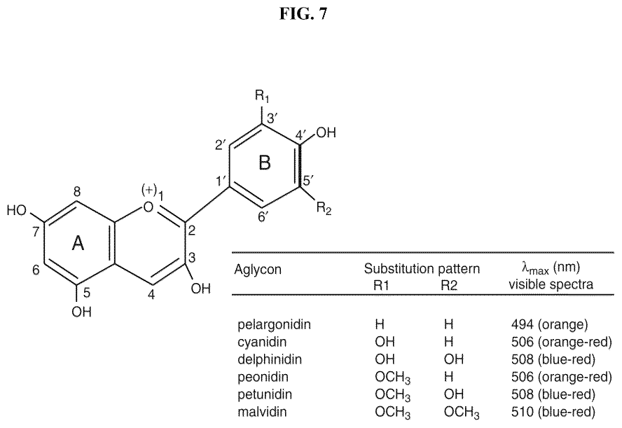

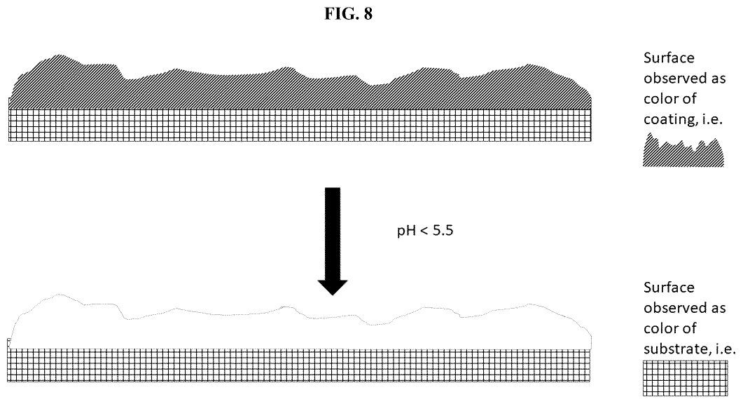

Anthocyanins, are natural colorimetric pH responsive material that are derived from different plants, fruits or vegetables. In some embodiments, the sensing agent is purified and isolated anthocyanins from different plant materials such as: red cabbage, black currant, chokeberry, onion, radish, peaches, and wild blueberries in order to identify the anthocyanin that yields the most prominent color change above and below pH 5.5., i.e., to identify the material or mixture of materials with the biggest intensity change and absorbance shift near pH 5.5. Anthocyanins, which are derivatized anthocyanidins, are, in embodiments the sensing agent, including but not limited to those listed below and shown in FIG. 7:
1. 3,3′,4′,5,7-Pentahydroxy-flavylium chloride (cyanidin)
2. 3,4′,5,7-Tetrahydroxy-3′-methoxyflavylium chloride (peonidin)
3. 3,4′,5,7-Tetrahydroxy-3′,5′-dimethoxyflavylium chloride (malvidin)
4. 3,5,7-Trihydroxy-2-(3,4,5,trihydroxyphenyl)-1-benzopyrylium chloride (delphinidin)
5. 3,3′4′,5,7-Pentahydroxy-5′-methoxyflavylium chloride petunidin)
6. 3,5,7-Trihydroxy-2-(4-hydroxyphenyl)-1-benzopyrilium chloride (pelargonidin)

Common anthocyanins (labelled at position 3 of anthocyanidins, also in embodiments, the present sensing agent) found in fruits and vegetables are listed in the table below:

TABLE 1

| | | CAS | |
|---|---|---|---|
| Common name | IUPAC name | Number | Formula |
| Pelargonidin-3- | 5,7-Dihydroxy-2-(4-hydroxyphenyl)-3- | 47684-27-5 | C21H21ClO10 |
| O-glucoside | {[(2S,3R,4S,5S,6R)-3,4,5-trihydroxy-6- | | |
| (Callistephin) | (hydroxymethyl)oxan-2-yl]oxy}-1λ4- | | |
| | chromen-1-ylium | | |
| Cyanidin 3-O- | (2S,3R,4S,5S,6R)-2-[2-(3,4- | 7084-24-4 | C21H21ClO11 |
| glucoside | dihydroxyphenyl)-5,7- | | |
| (Chrysanthemin) | dihydroxychromenylium-3-yl]oxy-6- | | |
| | (hydroxymethyl)oxane-3,4,5-triol | | |
| | chloride | | |
| Delphinidin 3-O- | (2S,3R,4S,5S,6R)-2-[5,7-dihydroxy-2- | 6906-38-3 | C21H21ClO12 |
| glucoside | (3,4,5-trihydroxyphenyl)chromenylium- | | |
| (Myrtillin) | 3-yl]oxy-6-(hydroxymethyl)oxane-3,4,5- | | |
| | triol chloride | | |
| Peonidin-3-O- | 5,7-Dihydroxy-2-(4-hydroxy-3- | 68795-37-9 | C22H23ClO11 |
| glucoside | methoxyphenyl)-3-{[(2S,3R,4S,5S,6R)- | | |
| | 3,4,5-trihydroxy-6- | | |
| | (hydroxymethyl)oxan-2-yl]oxy}-1λ4- | | |
| | benzopyran-1-ylium | | |
| Petunidin-3-O- | (2S,3R,4S,5S,6R)-2-[2-(3,4-dihydroxy-5- | 6988-81-4 | C22H23ClO12 |
| glucoside | methoxyphenyl)-5,7- | | |
| | dihydroxychromenylium-3-yl]oxy-6- | | |
| | (hydroxymethyl)oxane-3,4,5-triol | | |
| Malvidin 3-O- | 5,7-Dihydroxy-2-(4-hydroxy-3,5- | 7228-78-6 | C23H25ClO12 |
| glucoside | dimethoxyphenyl)-3-{[(2S,3R,4S,5S,6R)- | | |
| (Oenin) | 3,4,5-trihydroxy-6- | | |
| | (hydroxymethyl)oxan-2-yl]oxy}-1λ4- | | |
| | benzopyran-1-ylium | | |

Total anthocyanin mass (regardless of type of anthocyanidin), and their acylation derivatization can impact color changes observed at different pH by unassisted eye. The table below shows the mass of anthocyanins, fiber acylation, and sugar content, as well as calculations of anthocyanin content upon removal of fibers (column 4), and upon removal of both sugar and fiber (column 7) in the materials.

TABLE 2

| | | | | | | Anthocyanin |
|---|---|---|---|---|---|---|
| | | Insoluble | Anthocyanin | | | in fiberless, |
| | Anthocyanins | Fiber | in fiberless | | Sugar | sugarless |
| | in dried mass | content of | dried mass | Acylation | content in | dried mass |
| | % w/w | dried mass | % w/w | content | dried mass | % w/w |
| Wild blueberry | 4.4% | 18-22% | 5.4-5.6% | 13% | 40-60% | 10-24% |
| (Vaccinium | | | | | | |
| angustifolium) | | | | | | |
| (Wymann's) | | | | | | |
| Chokeberry | 5.2% | 22-28% | 6.7-7.2% | 0% | 25-30% | 9.8-12% |
| (Aronia | | | | | | |
| melanocarpa) | | | | | | |
| (Bobica) | | | | | | |
| Black currant | 2.1% | 25-30% | 2.8-3.0% | 1% | 35-40% | 5.3-10% |
| (Ribes nigrum) | | | | | | |
| (Bobica) | | | | | | |
| Red cabbage | 3.6% | 20-28% | 4.5-5.0% | 85% | 40-50% | 9.0-16% |
| (Brassica | | | | | | |
| oleracea var. | | | | | | |
| capitata f. | | | | | | |
| rubra) (Fluxia) | | | | | | |
| Red onion | 0.4% | 12-20% | 0.5% | 77% | 35-40% | 0.8-1.1% |
| (Allium cepa) | | | | | | |
| (Holy Natural) | | | | | | |
| Red radish | 2.3% | 35-40% | 3.5-3.8% | 100% | 40-50% | 9.2-23% |
| (Raphanus | | | | | | |
| raphanistrum | | | | | | |
| subsp. Sativus) | | | | | | |
| (Suncore) | | | | | | |

The anthocyanin was then extracted for measuring and refining the color change in solution by as unassisted eye as described hereafter in the examples below.

The disclosure also provides kits that can detect the presence or absence of a dental caries, and/or cavity or cavities, or precursor thereof, as described in the embodiments herein. A typical kit of the disclosure comprises various reagents including, for example, one or more devices (e.g., a device, including a support, a substrate, and a sensing agent, as disclosed herein) useful to detect the presence or absence of a dental caries, and/or cavity or cavities, or precursor thereof. The kit can include a visualization surface to detect a color change by an unassisted eye. The kit can further comprise materials necessary for the evaluation, including a dental pick, toothpick, paper point, rubber tip stimulator, a gum stimulator, dental floss, gel form, tray, or test strip, and the like. The kit can further comprise a label or printed instructions instructing the use of described reagents, including a reference color card. The kit can further comprise a treatment to be tested.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

This disclosure is further illustrated by the following non-limiting examples.

### EXAMPLES

### Example 1: Selection of Dye to Yield Choice for Yes No Transition to an Unassisted Eye at pH 5.5

In the experiments of this example, a fruit/vegetable extract was retrieved via dissolution in water and filtration to remove fibers. The fiberless solution was then adjusted for a targeted pH level (3-8.5) using 0.1M NaOH. Manufactured commercial powders or natural fruits/vegetables were then used to produce the initial extract (see Table 2 above). Thereafter, the different solutions were assessed for the most observable color change by an unassisted eye.

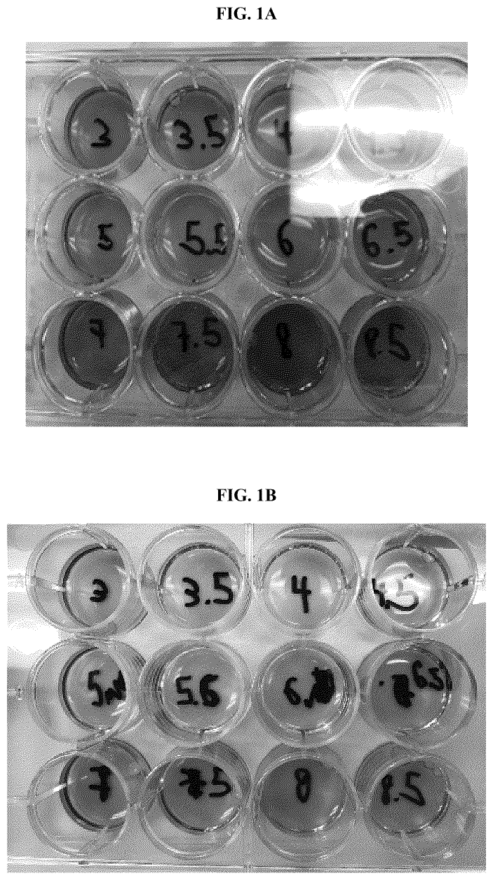

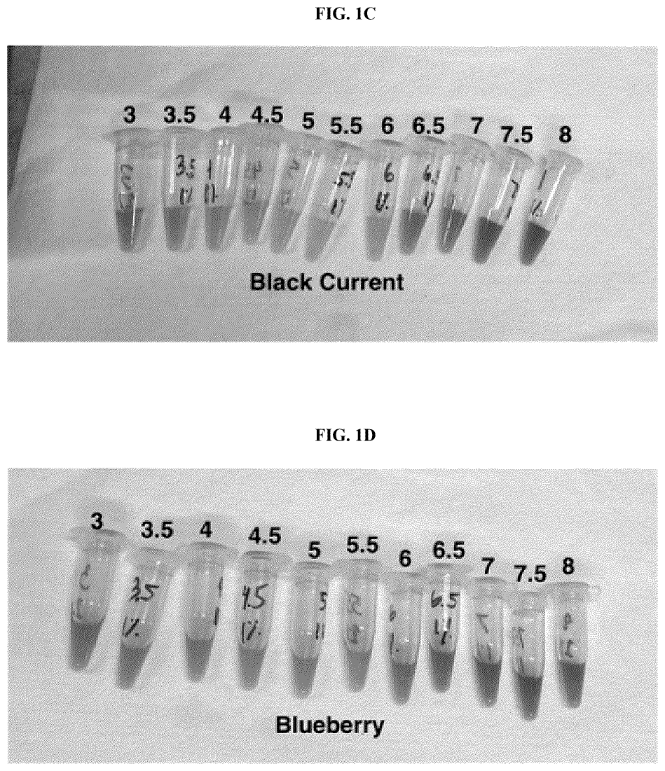

FIG. TA, FIG. 1B, FIG. 1C, FIG. 1D, FIG. 1E, and FIG. 1F are images showing radish (FIG. TA), onion (FIG. 1B), black current (FIG. 1C), blueberry (FIG. 1D), red cabbage (FIG. 1E), and chokeberry (FIG. 1F) extract solutions containing anthocyanin at pH levels of 3.0, 3.5, 4.0, 4.5, 5.0, 5.5, 6.0, 6.5, 7.0, 7.5, 8.0, and 8.5.

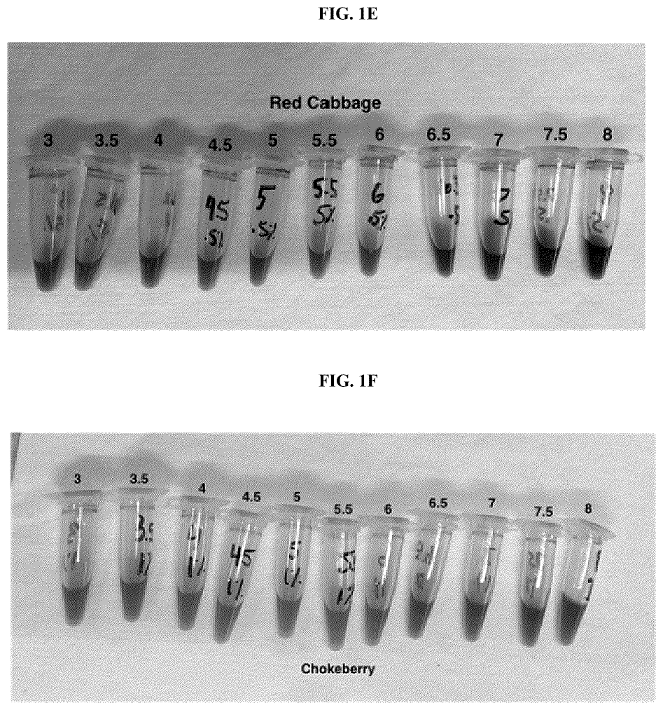

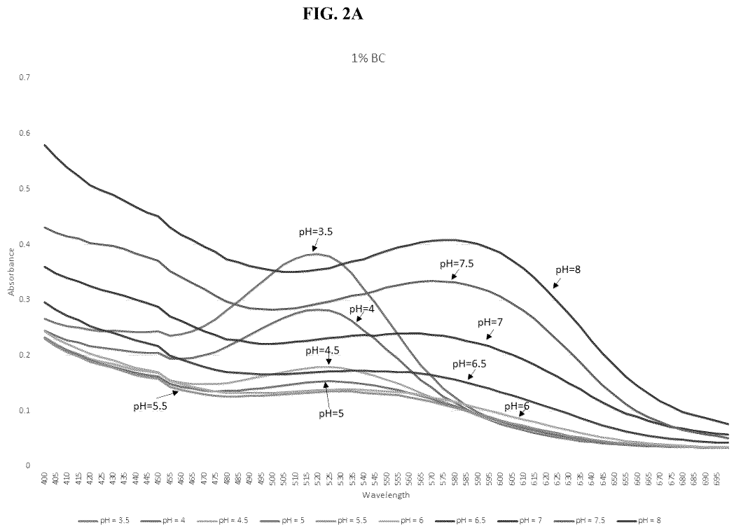

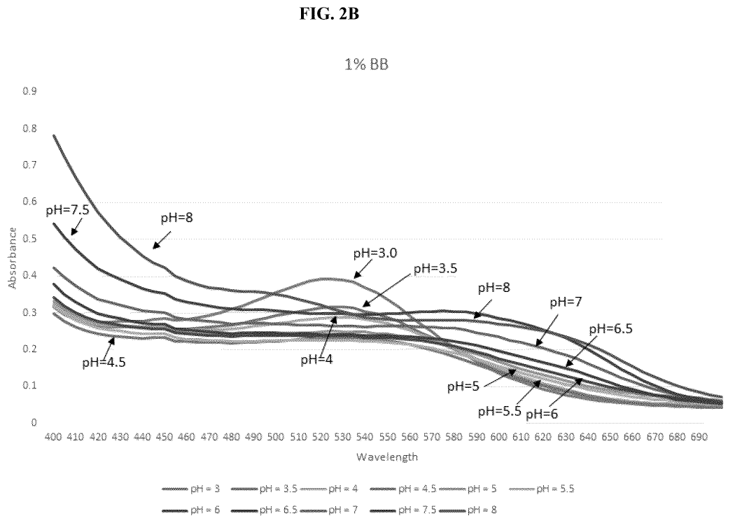

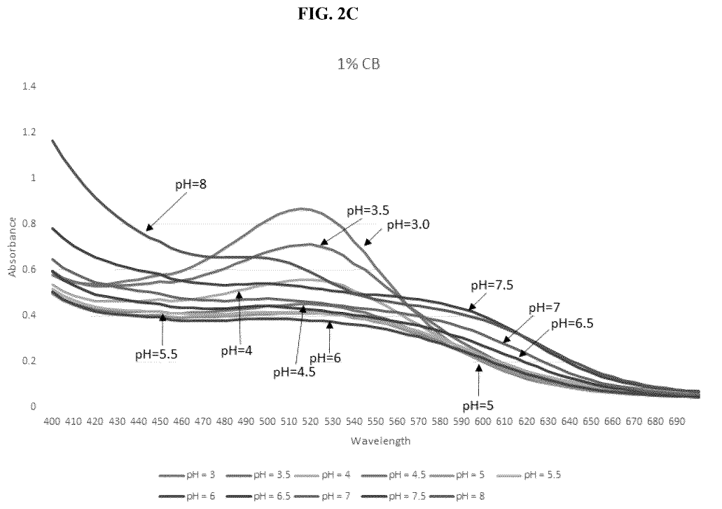

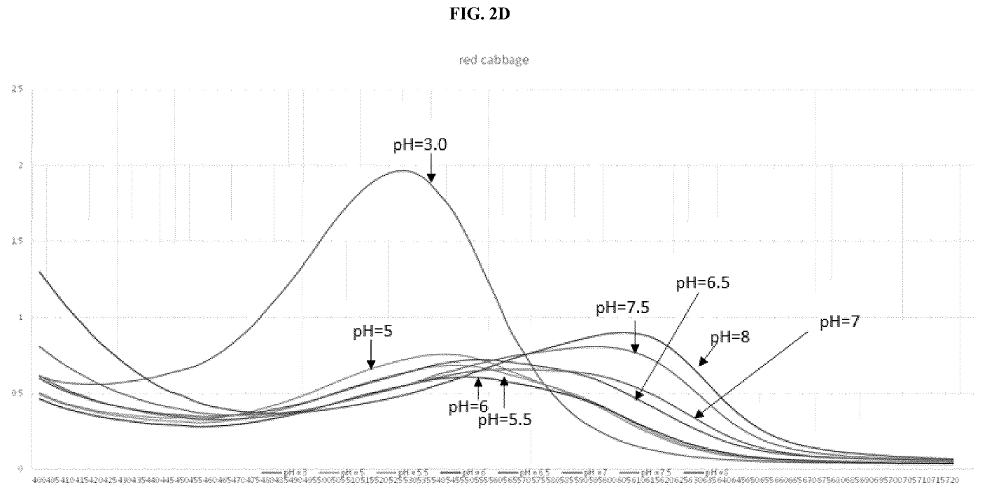

Among all of the tested fruits and vegetables, the most significant color change was observed at a specific pH level of 5.5, which was observed in the blueberries and red cabbage samples. This pH level of 5.5 represents active tooth demineralization (e.g., caries and/or cavity, tooth decay). The observed color change in these experiments is dependent on the interplay of various parameters including intensity (correlated to the amount anthocyanin), breadth of wavelength shift at or near pH 5.5 (likely correlated to acylation content), and the processing conditions. FIG. 2A, FIG. 2B, FIG. 2C, and FIG. 2D shows graphs of absorbance spectra of the different anthocyanins solutions at different wavelengths as correlated to pH levels of 3-8. The graphs in FIG. 2A, FIG. 2B, FIG. 2C, and FIG. 2D show the wavelength absorbance shifts for different pH. The spectra in this experiment were taken at 0.5% and 1% concentration of fiber-less dried mass. Wild blueberry (BB) (FIG. 2C) contains ˜5.5% w/w anthocyanin in fiber-less dried mass (see Table 2 above). Thus, a 1% solution of this dried mass of BB contains 0.05% anthocyanins in solution (i.e., 1×5.5% ═0.05% anthocyanins in solution) of the spectrum (FIG. 2). Similarly, Chokeberry (CB) (FIG. 2B) contains ˜7.0% w/w anthocyanin in fiber-less dried mass (see Table 2 above). Thus a 1% solution of this dried mass of CB contains 1×6.9%=0.069% anthocyanins in solution of the spectrum, similar to BB. BB however contains 13% acylation content while chokeberry has none. Based on the experiments disclosed herein, this impacts the absorbance spectra at the relevant pH threshold, and analogously, enhances a ready visualization by an unassisted eye of the color change.

As mentioned above, FIG. 2A, FIG. 2B, FIG. 2C, and FIG. 2D are graphs showing the absorbance spectra of 1% solution of black current (BC) (FIG. 2A), chokeberry (CB) (FIG. 2B), wild blueberry (BB) (FIG. 2C), and 0.5% red cabbage (RC) (FIG. 2D) at different wavelengths and pH. BB shows a shift in wavelength between pH 6.9 to 5.3, which are visible to the unassisted eye. The chokeberry (CB) (FIG. 2B) shows change in intensity, however it does not show a shift in wavelength in this experiment, and thus a shift in color is not observable but the intensity is observable—when intensity lowers the color becomes transparent. The Blackcurrent (BC) does not show a change in wavelength between pH 6.5 and 5.5, but does show a significant reduction in absorbance which is indicating of becoming transparent.

### Example 2: Testing of Dye Concentration and Dye Layering Spacing to Achieve Maximum Visible Color Change Based on Fixed Volume of Saliva Buffered Solution

The experiments of this example showed that the most visible color change by an unassisted eye increased with the total anthocyanin concentration.

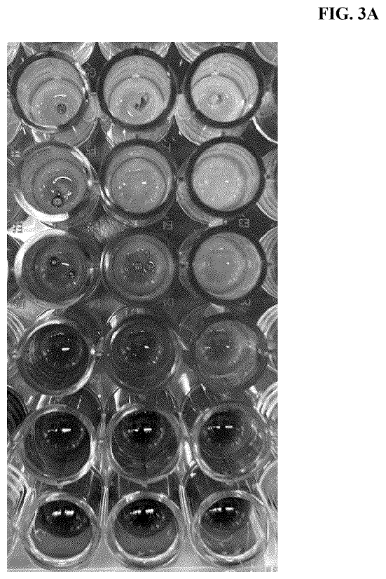

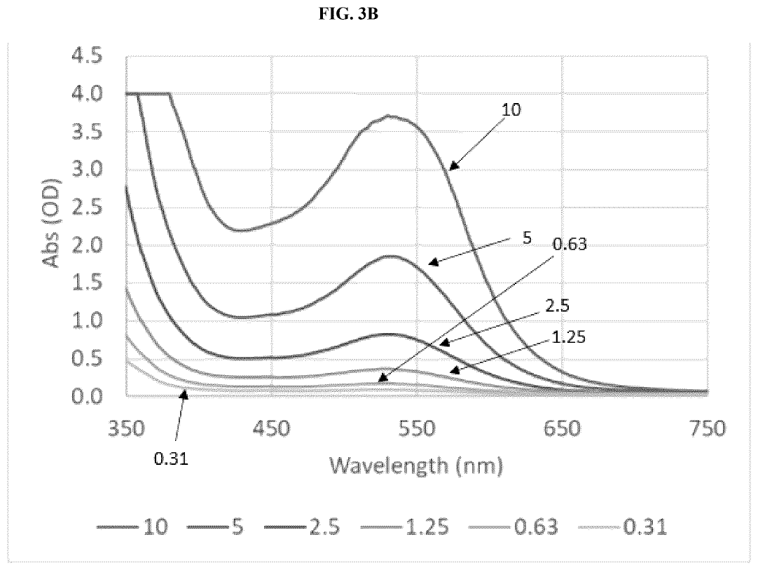

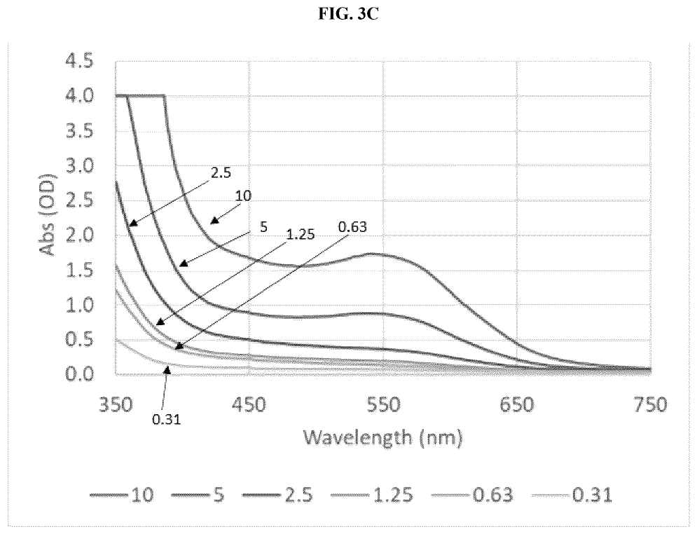

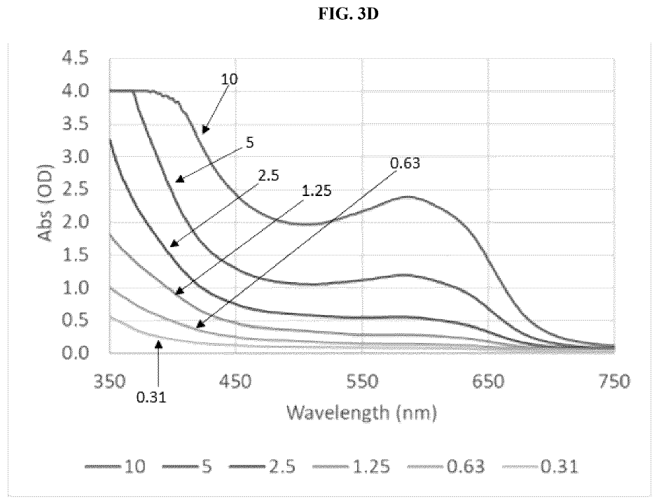

In the experiments of this example, Wymann's wild blueberry (BB) powder weighing 9.87 grams was placed into a glass beaker to which 40 mL DI water was added. The solution was then heated to 65° C. for 0.5 hrs. The solution was thereafter filtered through a Corning disposable vacuum filtration system (0.22 μm PES). The filtrate was frozen at −80° C., and lyophilized in Labconco Freezone lyophilizer for 3 days with minimal vacuum reaching 0.18 Bar. 0.50 g of dried mass was then added with 0.50 mL of water to obtain 50% solution. The material was further diluted accordingly to obtain lower concentrations of materials. 100 μL of different concentrated solutions ranging from 10% to 0.31% were added to a 96 well plate and subsequently read by a spectrophotometer to generate absorbance spectra. FIG. 3A, FIG. 3B, FIG. 3C, and FIG. 3D are images and graphs showing wild blueberry solutions at different concentrations. FIG. 3A is a visual image of a 96 well plate showing different concentrations of wild blueberry (from left to right, pH 3.3, 8.0 and 5.0; concentrations of solutions from top to bottom are 0.31%, 0.63%, 1.25%, 2.5%, 5.0%, and 10%.). In this experiment, a 100 μL of sample was placed in 96 well plate to have a height of 0.3 cm (i.e., L=0.3 in A=ecL). FIG. 3B is a graph showing the absorbance spectra of wild blueberry at a pH level of 3.0. FIG. 3C is a graph showing the absorbance spectra of wild blueberry at a pH level of 5.0. FIG. 3D is a graph showing the absorbance spectra of wild blueberry at a pH level of 8.0. FIG. 3B, FIG. 3C, and FIG. 3D are absorption spectra of BB at various concentrations in terms of percent. In these experiments, 100 μL of sample was placed in 96 well plate to have a height of 0.3 cm (i.e., L=0.3 cm in A=ecL). These experiments show the difference in absorbance in all measured pH was increasing with the increase in concentration. Accordingly, a higher concentration of the anthocyanins, as shown experimentally herein, results in a significant observable color change.

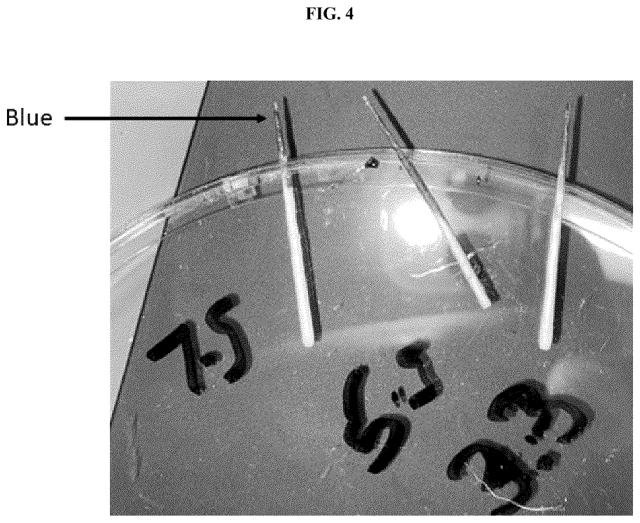

To investigate concentration levels above 10%, a dip coating technique was used (FIG. 4). In these experiments, the dip coating was performed with highest concentrations of solutions (about ˜50%). Coarse paper points were dipped in pH adjusted solutions for 1-3 seconds. The coarse paper points were then dried in air for 5 minutes. The dried paper points were then added with 5-7 μL of phosphate buffered solutions at pH 3.3, 5.5, and 7.5 (FIG. 4).

The filtrate can be further concentrated with anthocyanins by removing sugar to further amplify the color visualization. Solubilizing the above powdered material in methanol allows for extraction of anthocyanins in much greater quantities by removing sugar. The solubility of anthocyanins is similar in both methanol and water as seen in Table 3 below. However, sucrose is far more soluble in water than in methanol (210 g/100 mL in water vs 1 g/100 ml in methanol). This difference in solubility is exploited to achieve a higher concentration of anthocyanins in the solution. A 50% solution devoid of sugar can have up to 12%% anthocyanin content (i.e., the maximum anthocyanin content in fiber and sugar removed mass is 24%, thus 50% of this solution has 12% anthocyanins).

TABLE 3
Mole Fraction Solubility x₁, Activity Coefficient y,
and Relative Deviation (x₁ − x_{1calc})/x₁) of Delphinidin
in Pure Solvents from T = (298.15 to 343.15) K

| | | | |
|---|---|---|---|
| T/K | 10⁸ (x₁ ± SD) | γ | (x₁ − x_{1calc})/x₁ |

Water

| | | | |
|---|---|---|---|
| 298.15 | 53.53 ± 0.06 | 415.64 | −0.09 |
| 303.15 | 63.87 ± 0.03 | 451.54 | 0.07 |

Methanol

| | | | |
|---|---|---|---|
| 298.15 | 58.61 ± 0.01 | 378.16 | −0.01 |
| 303.15 | 66.24 ± 0.04 | 430.22 | −0.06 |

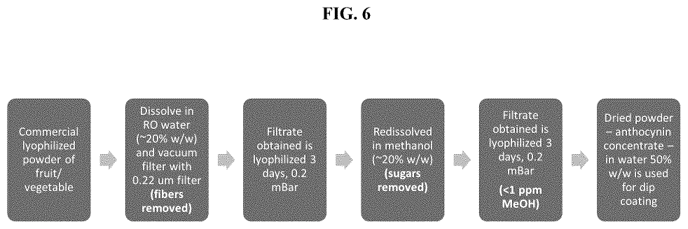

The process flow of anthocyanin concentration is shown in FIG. 6.

### Example 3: Introduction of Black Background to Visualize Color Changes Under any Lighting Conditions

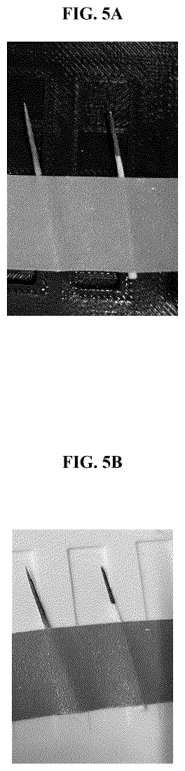

In the experiments of this example, different background colors enhance the visualization of the different colors by an unassisted eye. In these experiments (FIG. 5A and FIG. 5B), paper points with marginally different pHs (thus colors) are shown. FIG. 5A shows the chokeberry dye at pH levels of 5.5 and 6.5, against a black background. FIG. 5B shows the same paper points against a white background. The differing colors in FIG. 5B are not visible to a naked eye. However, as shown in FIG. 5A, the small difference in colors are readily visible with a black background. The usage of a black background absorbs all colors (all wavelengths), and a white background reflects all wavelengths, which ultimately leads to an increase in signal to noise ratio. Thus, the same paper points with marginal variation in colors/pHs are visualized (seen) with black background, but not white background.

### Example 4: Summary of the Surface Color Change

In the experiments of this example, a summary of the color change on the surface, as described herein, is shown in FIG. 8. Without wishing to be bound by theory, this image shows a color change on a surface from dark grey to transparent/translucent when the pH level is less than 5.5. The color change, without wishing to be bound by theory, can also be from yellow to transparent/translucent when the pH level is less than 5.5. The color change visualizes an underlying substrate. When the pH is less than 5.5, the colored substrate and colored dye become transparent/translucent, allowing for visualization of the substrate. This reaction also reverses with colorless dye that becomes colored and blocks the substrate color at pH<5.5. FIG. 8 shows a “one-color-change” implementation. For example, the user sees one color, then another color at pH 5.5, but one of the colors is the substrate. In FIG. 8, the color change is in the surface from colored to colorless, or substantially colorless, thereby exposing the substrates blueness in this example.

Visually, the signal in FIG. 8 is the reflectivity of the surface. The reflectivity of the surface has two components: the reflectivity of the sensing agent, and the reflectivity of the substrate. The reflectivity of the sensing agent, and the reflectivity of the substrate can be visualized independently or in combination.

All of the features disclosed herein may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A device for detection of a presence or absence of a dental caries, and/or cavity or cavities, and/or cavity activity risk, and/or caries risk, or precursor thereof, comprising:
(a) a support, the support being configured for grasping;
(b) a substrate attached to or layered upon the support and being suitable for absorption or containing a sensing agent and detection of the sensing agent without interference; and
(c) a sensing agent, the sensing agent being suitable for providing a color change which is indicative of a pH value associated with the presence or absence of a dental caries, and/or cavity or cavities, risk for caries, caries activity, or precursor thereof.

2. The device of claim 1, wherein the device is suitable for contacting a surface of a tooth.

3. The device of claim 2, wherein the contacting is interproximal.

4. The device of any one of claims 1-3, wherein the support is or comprises a dental pick, toothpick, paper point, rubber tip stimulator, a gum stimulator, dental floss, gel form, tray, or test strip.

5. The device of any one of claims 1-4, wherein the sensing agent is substantially absorbed into the substrate.

6. The device of any one of claims 1-5, wherein the sensing agent covers substantially the entire external surface of the substrate, optionally wherein the sensing agent is contained within the substrate.

7. The device of any one of claims 1-6, wherein the substrate is or comprises a biologically compatible polymer matrix or biopolymer matrix material.

8. The device of any one of claims 1-7, wherein the substrate is or comprises one or more of a fibroin, silk fibroin, actin, alginate, collagen, catenin, claudin, coilin, elastin, elaunin, extensin, fibrillin, keratin, lamin, laminin, tublin, viral structural protein, zein protein, polyethylene oxide, polyethylene glycol, fibronectin, polyaspartic acid, polylysine, alginate, chitosan, chitin, hyaluronic acid, pectin, polycaprolactone, polylactic acid, polyglycolic acid, polyhydroxyalkanoate, silicone, dextran, polyanhydride, gelatin, gel, lignin, cellulose, polyanilines, polypyrroles, a plastic polymer, a thermoforming polymer, a 3D printed polymer, and/or optionally a gel mixed with at least one material selected from fibroin, silk fibroin, actin, alginate, collagen, catenin, claudin, coilin, elastin, elaunin, extensin, fibrillin, keratin, lamin, laminin, tublin, viral structural protein, zein protein, polyethylene oxide, polyethylene glycol, fibronectin, polyaspartic acid, polylysine, alginate, chitosan, chitin, hyaluronic acid, pectin, polycaprolactone, polylactic acid, polyglycolic acid, polyhydroxyalkanoate, silicone, dextran, polyanhydride, gelatin, gel, lignin, cellulose, polyanilines, polypyrroles, a plastic polymer, a thermoforming polymer, and a 3D printed polymer.

9. The device of any one of claims 1-8, wherein the sensing agent is or comprises a silk fibroin derived from a silkworm, or a spider, or wherein the substrate is or comprises a naturally occurring carbonaceous material, optionally wherein the naturally occurring carbonaceous material is activated carbon.

10. The device of claim 9, wherein the silkworm is Bombyx mori.

11. The device of any one of claims 1-10, wherein the substrate is modified to absorb the sensing agent.

12. The device of any one of claims 1-11, wherein the substrate is modified to provide an unobfuscated color signal.

13. The device of any one of claims 1-12, wherein the substrate is modified to reduce porosity relative to an unmodified form.

14. The device of any one of claims 1-13, wherein the substrate is modified to reduce porosity and increase external surface exposure to sensing agent, relative to an unmodified form.

15. The device of any one of claims 1-14, wherein the sensing agent is or comprises a food-derived agent; optionally wherein the food-derived agent is or comprise a food coloring compound, optionally wherein the sensing agent is or comprises a food extract of food color compound, optionally wherein the food compound or food extract compound is selected from one or more of a carotenoids, chlorophyll, anthocyanin, and turmeric, or a derivative thereof; optionally wherein the food coloring compound or food extract compound is a beta-carotene molecule, anthocyanin, carminic acid, or a derivative thereof.

16. The device of any one of claims 1-15, wherein the sensing agent is or comprises an anthocyanin or derivative thereof.

17. The device of claim 16, wherein the anthocyanin or derivative thereof is selected from a flower, a herb, a food and/or spice source, a fermented beverage; or wherein the anthocyanin or derivative thereof is selected from Pelargonidin-3-O-glucoside (Callistephin), Cyanidin 3-O-glucoside (Chrysanthemin), Delphinidin 3-O-glucoside (Myrtillin), Peonidin-3-O-glucoside, Petunidin-3-O-glucoside, Malvidin 3-O-glucoside (Oenin), and combinations thereof.

18. The device of any one of claims 1-15, wherein the sensing agent is or comprises an anthocyanidin or derivative thereof, a curcumin or derivative thereof, or a porhyrin, or derivative thereof.

19. The device of claim 18, wherein the anthocyanidin or derivative thereof is selected from one or more of a flower, a herb, a food and/or spice source, a fermented beverage; or wherein the anthocyanidin or derivative thereof is selected from 3,3′,4′,5,7-Pentahydroxy-flavylium chloride (cyanidin), 3,4′,5,7-Tetrahydroxy-3′-methoxyflavylium chloride (peonidin), 3,4′,5,7-Tetrahydroxy-3′,5′-dimethoxyflavylium chloride (malvidin), 3,5,7-Trihydroxy-2-(3,4,5,trihydroxyphenyl)-1-benzopyrylium chloride (delphinidin), 3,3′4′,5,7-Pentahydroxy-5′-methoxyflavylium chloride (petunidin), 3,5,7-Trihydroxy-2-(4-hydroxyphenyl)-1-benzopyrilium chloride (pelargonidin), and combinations thereof.

20. The device of any one of claims 1-19, wherein the sensing agent is solubilized in a solution and present in the substrate at a concentration of at least 0.1%, at least 0.2%, at least 0.3%, at least 0.4%, at least 0.5%, at least 0.6%, at least 0.7%, at least 0.8%, at least 0.9%, at least 1.0%, at least 1.25%, at least 1.50%, at least 1.75%, at least 2.0%, at least 2.25%, at least 2.50%, at least 5.0%, at least 10.0%, at least 11.0%, at least 12.0%, at least 13.0%, at least 14.0%, at least 15.0% at least 20.0%, at least 25.0%, at least 30.0%, at least 35.0%, at least 40.0%, at least 45.0%, or at least 50.0%.

21. The device of claim 20, wherein the sensing agent is present in the substrate at a concentration from at least 0.1% to at least 10%.

22. The device of claim 20, wherein the sensing agent is present in the substrate at a concentration from at least 0.3% to at least 0.75%.

23. The device of claim 20, wherein the sensing agent is present in the substrate at a concentration from at least 1.0% to at least 5.0%.

24. The device of claim 20, wherein the sensing agent is present in the substrate at a concentration from at least 5.0% to at least 10.0%.

25. The device of claim 20, wherein the sensing agent is present in the substrate at a concentration from at least 10.0% to at least 50.0%.

26. The device of any one of claims 20-25, wherein the sensing agent is solubilized in methanol, ethanol, sucrose, water mixtures, or water, or wherein the sensing agent is solubilized in a sugar, a carbohydrate, or a PEG-based material.

27. The device of any one of claims 20-26, wherein the solution is devoid of sugar.

28. The device of claim 27, wherein the content of the sensing agent in the solution devoid of sugar is a concentration of at least 0.1%, at least 0.2%, at least 0.3%, at least 0.4%, at least 0.5%, at least 0.6%, at least 0.7%, at least 0.8%, at least 0.9%, at least 1.0%, at least 1.25%, at least 1.50%, at least 1.75%, at least 2.0%, at least 2.25%, at least 2.50%, at least 5.0%, at least 10.0%, at least 11.0%, at least 12.0%, at least 13.0%, at least 14.0%, or at least 15.0%.

29. The device of claim 28, wherein the content of the sensing agent in the solution devoid of sugar is at a concentration of at least 0.1% to 12%.

30. The device of any one of claims 1-29, wherein the sensing agent is extracted from one or more food and/or spice sources and concentrated.

31. The device of any one of claims 1-30, wherein the sensing agent is extracted from one or more food and/or spice sources by filtering and concentrating a lyophilized powder of the one or more food and/or spice sources, optionally wherein the device detects the presence or absence of a dental caries, and/or cavity or cavities, for all surfaces of the teeth of a subject.

32. The device of any one of claims 1-31, wherein the device is suitable for displaying a color change of a first color to a second color that indicates a specific pH value.

33. The device of any one of claims 1-31, wherein the device is suitable for displaying a color change of a first color to a second color that does not indicate a specific pH value.

34. The device of claim 32 or 33, wherein the first color is a primary color.

35. The device of claim 34, wherein the first color is red, yellow, or blue.

36. The device of claim 32 or 33, wherein the first color is a secondary color.

37. The device of claim 36, wherein the first color is orange, green, or violet.

38. The device of claim 32 or 33, wherein the first color is a tertiary color.

39. The device of claim 38, wherein the first color is red-orange, yellow-orange, yellow-green, blue-green, blue-violet, or red-violet.

40. The device of claim 32 or 33, wherein the second color is a primary color.

41. The device of claim 40, wherein the second color is red, yellow, or blue.

42. The device of claim 32 or 33, wherein the second color is a secondary color.

43. The device of claim 42, wherein the second color is orange, green, or violet.

44. The device of claim 32 or 33, wherein the second color is a tertiary color.

45. The device of claim 44, wherein the second color is red-orange, yellow-orange, yellow-green, blue-green, blue-violet, or red-violet.

46. The device of any one of claims 1-31, wherein the device is suitable for displaying a color change of colored to colorless, or substantially colorless, that indicates a specific pH value.

47. The device of any one of claims 1-31, wherein the device is suitable for displaying a color change of colorless, or substantially colorless, to colored that indicates a specific pH value.

48. The device of any one of claims 1-31, wherein the device is suitable for displaying a color change of colored to colorless, or substantially colorless, that does not indicate a specific pH value.

49. The device of any one of claims 1-31, wherein the device is suitable for displaying a color change of colorless, or substantially colorless, to colored that does not indicate a specific pH value, or optionally wherein the device is suitable for displaying a color change of colorless that is revealing or disappearing of a pattern that is embedded on the substrate, wherein stripes disappear and/or appear when the test is positive or negative.

50. The device of any one of claims 1-49, wherein the sensing agent has a high absorbance.

51. The device of claim 50, wherein the sensing agent has a high absorbance and is applied to the substrate in a monolayer, a bilayer, a solution, or on a surface.

52. The device of any one of claims 1-49, wherein the sensing agent has a low absorbance, optionally wherein the sensing agent is applied to the substrate in solution, or on a surface.

53. The device of claim 52, wherein the sensing agent has a low absorbance and is applied to the substrate in a multilayer, optionally about 3, or about 4, or about 5, or about 6, or about 7, or about 8, or about 9, or about 10 layers.

54. The device of any one of claims 1-53, wherein the device is suitable for detecting color change by an unassisted eye when placed over a visualization surface, the visualization surface being black, reflective, matted, semi-reflective, or a color which is distinct from the color which is indicative of the pH value.

55. The device of claim 54, wherein the visualization surface is a primary color.

56. The device of claim 55, wherein the visualization surface is red, yellow, or blue.

57. The device of claim 54, wherein the visualization surface is a secondary color.

58. The device of claim 56, wherein the visualization surface is orange, green, or violet.

59. The device of claim 54, wherein the visualization surface is a tertiary color.

60. The device of claim 59, wherein the visualization surface is red-orange, yellow-orange, yellow-green, blue-green, blue-violet, or red-violet.

61. The device of any one of claims 1-60, wherein the device is suitable for detecting pH in saliva.

62. The device of any one of claims 1-61, wherein the device is suitable for detecting pH in gingival crevicular fluid.

63. The device of any one of claims 1-62, wherein the dental caries, and/or cavity or cavities, or precursor thereof is not readily detectable with visual-tactile examination.

64. The device of any one of claims 1-63, wherein the device is suitable for at-home detection.

65. The device of any one of claims 1-64, wherein the device is suitable for minimally invasive detection.

66. The device of any one of claims 1-65, wherein the device provides a signal that is visualizable by an unassisted eye.

67. The device of any one of claims 1-66, wherein the device augments or supplants a cavity detection method, optionally wherein the device augments or supplants radiographic detection, or wherein the device supplants Quantitative Light-induced Fluorescence (QLF), Fibre-optic Transillumination (FOTI), Electrical Conductance (EC), fluorescence or dyed particles/nanoparticles, bitewing radiography periapical radiography, panoramic radiography, or cone beam computed tomography (“CBCT”).

68. The device of any one of claims 1-67, wherein the device identifies an active caries and/or cavity, or a lack of an active caries and/or cavity.

69. The device of any one of claims 1-68, wherein the pH value is about 5.5.

70. The device of any one of claims 1-68, wherein the pH value is not about 5.5.

71. The device of any one of claims 1-68, wherein the pH value is about 5.5 and indicative of the presence of a dental caries, and/or cavity or cavities, or precursor thereof, and/or a risk of developing dental caries.

72. The device of any one of claims 1-68, wherein the pH value is not about 5.5 and indicative of the absence of a dental caries, and/or cavity or cavities, or precursor thereof.

73. The device of any one of claims 1-72, wherein the device detects demineralization, optionally of calcium, fluoride, and/or phosphate ions, optionally from enamel and/or dentin, optionally of enamel producing proteins and/or periodontal ligament degrading enzymes, optionally wherein the dentin and enamel producing proteins are selected from collagen and amelogenin, optionally wherein the dentin or periodontal ligament degrading enzyme is collagenase, optionally wherein the periodontal ligament degrading enzyme differentiates between deep and early stage cavities, optionally wherein the device detects demineralization of a catabolic enzyme, and optionally wherein the catabolic enzyme is a protease, sucrase, or sucrose synthase.

74. The device of any one of claims 1-72, wherein the device detects a lack or an absence of demineralization, optionally of calcium, fluoride, and/or phosphate ions, optionally from enamel and/or dentin.

75. The device of any one of claims 1-74, wherein the device detects activity of acid-secreting bacteria, optionally wherein the device increases the activity of acid-secreting bacteria.

76. The device of claim 75, wherein the device detects activity of Streptococci mutans, Streptococcus sobrinus, and/or Lactobacilli spp.

77. The device of any one of claims 1-74, wherein the device detects lack of activity of acid-secreting bacteria.

78. The device of claim 77, wherein the device detects lack of activity of Streptococci mutans, Streptococcus sobrinus, and/or Lactobacilli spp., optionally wherein the device detects bacterial acid secretion after intentional activation of bacterial acid secretion, optionally wherein the device increases bacterial acid secretion and/or bacterial activity.

79. The device of any one of claims 1-78, wherein the device detects a dental lesion.

80. The device of any one of claims 1-79, wherein the device distinguishes between active and arrested lesions.

81. The device of any one of claims 1-80, wherein the device detects an incipient dental lesion.

82. The device of any one of claims 1-81, wherein the device detects a reversible dental lesion, optionally wherein the device detects the depth of the dental lesion.

83. The device of any one of claims 1-82, wherein the device detects a remineralizable dental lesion.

84. A method of detecting a presence or absence of a dental caries, and/or cavity or cavities, risk for caries, or caries associated with bacterial acid production, or precursor thereof, comprising:
(a) contacting a surface of a tooth with the device of any one of claims 1-83, or with the sensing agent of any one of claims 1-32, 34-47, or 50-83, or a support and/or substrate comprising the sensing agent of any one of claims 1-32, 34-47, or 50-83; and
(b) determining a color change in the sensing agent, the color change being indicative of a pH value that is associated with the presence or absence of tooth demineralization, dental caries, risk for caries, caries associated bacterial acid production, and/or cavity or cavities, or precursor thereof, and/or risk of developing dental caries, and/or cavity or cavities presence or activity.

85. A method of detecting a presence or absence of a dental caries, and/or cavity or cavities, or precursor thereof, comprising:
(a) contacting a surface of a tooth with a sensing agent, or a support and/or substrate comprising the sensing agent, the sensing agent being configured to colorimetrically detect pH of saliva, plaque, or gingival crevicular fluid; and
(b) determining a color change in the sensing agent, the color change being indicative of a pH value that is associated with the presence or absence of tooth demineralization, dental caries, caries risk, caries activity level, caries associated bacterial activity, and/or cavity or cavities, or precursor thereof, and/or risk of developing dental caries, and/or cavity or cavities presence or activity.

86. The method of any one of claims 84-85, wherein the contacting is interproximal.

87. The method of any one of claims 84-86, wherein the dental caries, and/or cavity or cavities, or precursor thereof, is not readily detectable with visual-tactile examination.

88. The method of any one of claims 84-87, wherein the method provides at-home detection.

89. The method of any one of claims 84-88, wherein the method provides for minimally invasive detection.

90. The method of any one of claims 84-89, wherein the method provides a signal that is visualizable by an unassisted eye, optionally wherein the method provides a signal through a device, optionally wherein the method provides a signal through a phone application, optionally wherein the method provides a signal based on an image analysis algorithm, optionally wherein the method provides a signal that is visualizable by a software through taking an image of the device.

91. The method of any one of claims 84-90, wherein the method augments or supplants radiographic detection, optionally wherein the method augments or supplants Quantitative Light-induced Fluorescence (QLF), Fibre-optic Transillumination (FOTI) and Electrical Conductance (EC), bitewing radiography periapical radiography, panoramic radiography, or cone beam computed tomography (“CBCT”).

92. The method of any one of claims 84-91, wherein the method detects an active caries and/or cavity, or a lack of an active caries and/or cavity, optionally wherein the sensing agent detects the presence or absence of a dental caries, and/or cavity or cavities, for all surfaces of the teeth of a subject.

93. The method of any one of claims 84-92, wherein the pH value is about 5.5.

94. The method of any one of claims 84-93, wherein the pH value is not about 5.5.

95. The method of any one of claims 84-94, wherein the pH value is about 5.5 and indicative of the presence of a dental caries, and/or cavity or cavities, or precursor thereof, and/or a risk of developing dental caries.

96. The method of any one of claims 84-95, wherein the pH value is not about 5.5 and indicative of the absence of a dental caries, and/or cavity or cavities, or precursor thereof.

97. The method of any one of claims 84-96, wherein the method detects demineralization, optionally of calcium, fluoride, and/or phosphate ions, optionally from enamel and/or dentin, optionally of enamel producing proteins and/or periodontal ligament degrading enzymes, optionally wherein the dentin and enamel producing proteins are selected from collagen and amelogenin, optionally wherein the dentin or periodontal ligament degrading enzyme is collagenase, optionally wherein the periodontal ligament degrading enzyme differentiates between deep and early stage cavities, optionally wherein the device detects demineralization of a catabolic enzyme, and optionally wherein the catabolic enzyme is a protease, sucrase, or sucrose synthase.

98. The method of any one of claims 84-96, wherein the method detects a lack of demineralization, optionally of calcium, fluoride, and/or phosphate ions, optionally from enamel and/or dentin.

99. The method of any one of claims 84-98, wherein the method detects activity of acid-secreting bacteria, optionally wherein the method increases the activity of acid-secreting bacteria.

100. The method of claim 99, wherein the method detects activity of Streptococci mutans, Streptococcus sobrinus, and/or Lactobacilli spp.

101. The method of any one of claims 84-98, wherein the method detects lack of activity of acid-secreting bacteria.

102. The method of claim 101, wherein the method detects lack of activity of Streptococci mutans, Streptococcus sobrinus, and/or Lactobacilli spp., optionally wherein the method detects bacterial acid secretion after intentional activation of bacterial acid secretion, optionally wherein the method increases bacterial acid secretion and/or bacterial activity.

103. The method of any one of claims 84-102, wherein the method detects a dental lesion.

104. The method of any one of claims 84-103, wherein the method distinguishes between active and arrested lesions.

105. The method of any one of claims 84-104, wherein the method detects an incipient dental lesion.

106. The method of any one of claims 84-105, wherein the method detects a reversible dental lesion, optionally wherein the method detects the depth of the dental lesion.

107. The method of any one of claims 84-106, wherein the method detects a remineralizable dental lesion, or improving oral hygiene.

108. The method of any one of claims 84-107, wherein the support is or comprises a dental pick, toothpick, paper point, rubber tip stimulator, a gum stimulator, dental floss, gel form, tray, or test strip.

109. The method of any one of claims 84-108, wherein the sensing agent is substantially absorbed into the substrate.

110. The method of any one of claims 84-109, wherein the sensing agent covers substantially the entire external surface of the substrate, optionally wherein the sensing agent is contained within the substrate.

111. The method of any one of claims 84-110, wherein the substrate is or comprises a biologically compatible polymer matrix or biopolymer matrix material.

112. The method of any one of claims 84-111, wherein the substrate is or comprises one or more of a fibroin, silk fibroin, actin, alginate, collagen, catenin, claudin, coilin, elastin, elaunin, extensin, fibrillin, keratin, lamin, laminin, tublin, viral structural protein, zein protein, polyethylene oxide, polyethylene glycol, fibronectin, polyaspartic acid, polylysine, alginate, chitosan, chitin, hyaluronic acid, pectin, polycaprolactone, polylactic acid, polyglycolic acid, polyhydroxyalkanoate, silicone, dextran, polyanhydride, gelatin, gel, lignin, cellulose, polyanilines and polypyrroles, a plastic polymer, a thermoforming polymer, a 3D printed polymer, and/or optionally a gel mixed with at least one material selected from fibroin, silk fibroin, actin, alginate, collagen, catenin, claudin, coilin, elastin, elaunin, extensin, fibrillin, keratin, lamin, laminin, tublin, viral structural protein, zein protein, polyethylene oxide, polyethylene glycol, fibronectin, polyaspartic acid, polylysine, alginate, chitosan, chitin, hyaluronic acid, pectin, polycaprolactone, polylactic acid, polyglycolic acid, polyhydroxyalkanoate, silicone, dextran, polyanhydride, gelatin, gel, lignin, cellulose, polyanilines, polypyrroles, a plastic polymer, a thermoforming polymer, and a 3D printed polymer.

113. The method of any one of claims 84-112, wherein the sensing agent is or comprises a silk fibroin derived from a silkworm, or a spider.

114. The method of claim 113, wherein the silkworm is Bombyx mori.

115. The method of any one of claims 84-114, wherein the substrate is modified to absorb the sensing agent.

116. The method of any one of claims 84-115, wherein the substrate is modified to provide an unobfuscated color signal.

117. The method of any one of claims 84-116, wherein the substrate is modified to reduce porosity relative to an unmodified form.

118. The method of any one of claims 84-117, wherein the substrate is modified to reduce porosity and increase external surface exposure to sensing agent relative to an unmodified form.

119. The method of any one of claims 84-118, wherein the sensing agent is or comprises a food-derived agent.

120. The method of any one of claims 84-119, wherein the sensing agent is or comprises an anthocyanin or derivative thereof.

121. The method of claim 120, wherein the anthocyanin or derivative thereof is selected from a flower, a herb, a food and/or spice sources, a fermented beverage; or wherein the anthocyanin or derivative thereof is selected from Pelargonidin-3-O-glucoside (Callistephin), Cyanidin 3-O-glucoside (Chrysanthemin), Delphinidin 3-O-glucoside (Myrtillin), Peonidin-3-O-glucoside, Petunidin-3-O-glucoside, Malvidin 3-O-glucoside (Oenin), and combinations thereof.

122. The method of any one of claims 84-119, wherein the sensing agent is or comprises an anthocyanidin or derivative thereof, a curcumin or derivative thereof, or a porhyrin, or derivative thereof.

123. The method claim 122, wherein the anthocyanidin or derivative thereof is selected from one or more of a flower, a herb, a food and/or spice source, a fermented beverage; or wherein the anthocyanidin or derivative thereof is selected from 3,3′,4′,5,7-Pentahydroxy-flavylium chloride (cyanidin), 3,4′,5,7-Tetrahydroxy-3′-methoxyflavylium chloride (peonidin), 3,4′,5,7-Tetrahydroxy-3′,5′-dimethoxyflavylium chloride (malvidin), 3,5,7-Trihydroxy-2-(3,4,5,trihydroxyphenyl)-1-benzopyrylium chloride (delphinidin), 3,3′4′,5,7-Pentahydroxy-5′-methoxyflavylium chloride (petunidin), 3,5,7-Trihydroxy-2-(4-hydroxyphenyl)-1-benzopyrilium chloride (pelargonidin), and combinations thereof.

124. The method of any one of claims 84-123, wherein the sensing agent is solubilized in a solution and present in the substrate at a concentration of at least 0.1%, at least 0.2%, at least 0.3%, at least 0.4%, at least 0.5%, at least 0.6%, at least 0.7%, at least 0.8%, at least 0.9%, at least 1.0%, at least 1.25%, at least 1.50%, at least 1.75%, at least 2.0%, at least 2.25%, at least 2.50%, at least 5.0%, at least 10.0%, at least 11.0%, at least 12.0%, at least 13.0%, at least 14.0%, at least 15.0% at least 20.0%, at least 25.0%, at least 30.0%, at least 35.0%, at least 40.0%, at least 45.0%, or at least 50.0%.

125. The method of claim 124, wherein the sensing agent is present in the substrate at a concentration from at least 0.1% to at least 10%.

126. The method of claim 124, wherein the sensing agent is present in the substrate at a concentration from at least 0.3% to at least 0.75%.

127. The method of claim 124, wherein the sensing agent is present in the substrate at a concentration from at least 1.0% to at least 5.0%.

128. The method of claim 124, wherein the sensing agent is present in the substrate at a concentration from at least 5.0% to at least 10.0%.

129. The method of claim 124, wherein the sensing agent is present in the substrate at a concentration from at least 10.0% to at least 50.0%.

130. The method of any one of claims 124-129, wherein the sensing agent is solubilized in methanol, ethanol, sucrose, water mixtures, or water, or wherein the sensing agent is solubilized in a sugar, a carbohydrate, or a PEG-based material.

131. The method of any one of claims 124-130, wherein the solution is devoid of sugar.

132. The method of claim 131, wherein the content of the sensing agent in the solution devoid of sugar is a concentration of at least 0.1%, at least 0.2%, at least 0.3%, at least 0.4%, at least 0.5%, at least 0.6%, at least 0.7%, at least 0.8%, at least 0.9%, at least 1.0%, at least 1.25%, at least 1.50%, at least 1.75%, at least 2.0%, at least 2.25%, at least 2.50%, at least 5.0%, at least 10.0%, at least 11.0%, at least 12.0%, at least 13.0%, at least 14.0%, or at least 15.0%.

133. The method of claim 132, wherein the content of the sensing agent in the solution devoid of sugar is a concentration of at least 0.1% to 12%.

134. The method of any one of claims 84-133, wherein the sensing agent is extracted from one or more food and/or spice sources and concentrated.

135. The method of any one of claims 84-134, wherein the sensing agent is extracted from one or more food and/or spice sources by filtering and concentrating a lyophilized powder of the one or more food and/or spice sources.

136. The method of any one of claims 84-135, wherein the method is suitable for displaying a color change of a first color to a second color that indicates a specific pH value.

137. The method of any one of claims 84-135, wherein the method is suitable for displaying a color change of a first color to a second color that does not indicate a specific pH value.

138. The method of any one of claims 136 or 137, wherein the first color is a primary color.

139. The method of claim 138, wherein the first color is red, yellow, or blue.

140. The method of any one of claims 136 or 137, wherein the first color is a secondary color.

141. The method of claim 140, wherein the first color is orange, green, or violet.

142. The method of any one of claims 136 or 137, wherein the first color is a tertiary color.

143. The method of claim 142, wherein the first color is red-orange, yellow-orange, yellow-green, blue-green, blue-violet, or red-violet.

144. The method of any one of claims 136 or 137, wherein the second color is a primary color.

145. The method of claim 144, wherein the second color is red, yellow, or blue.

146. The method of any one of claims 136 or 137, wherein the second color is a secondary color.

147. The method of claim 146, wherein the second color is orange, green, or violet.

148. The method of any one of claims 136 or 137, wherein the second color is a tertiary color.

149. The method of claim 148, wherein the second color is red-orange, yellow-orange, yellow-green, blue-green, blue-violet, or red-violet.

150. The method of any one of claims 84-136, wherein the method is suitable for displaying a color change of colored to colorless, or substantially colorless, that indicates a specific pH value.

151. The method of any one of claims 84-136, wherein the method is suitable for displaying a color change of colorless, or substantially colorless, to colored that indicates a specific pH value.

152. The method of any one of claims 84-135, wherein the method is suitable for displaying a color change of colored to colorless, or substantially colorless, that does not indicate a specific pH value.

153. The method of any one of claims 84-135, wherein the method is suitable for displaying a color change of colorless, or substantially colorless, to colored that does not indicate a specific pH value.

154. The method of any one of claims 84-153, wherein the sensing agent has a high absorbance.

155. The method of claim 154, wherein the sensing agent has a high absorbance and is applied to the substrate in a monolayer, a bilayer, a solution, or on a surface.

156. The method of any one of claims 84-153, wherein the sensing agent has a low absorbance.

157. The method of claim 156, wherein the sensing agent has a low absorbance and is applied to the substrate in a multilayer, optionally about 3, or about 4, or about 5, or about 6, or about 7, or about 8, or about 9, or about 10 layers.

158. The method of any one of claims 84-157, wherein the method is suitable for detecting color change by an unassisted eye when placed over a visualization surface, the visualization surface being black, reflective, matted, semi-reflective, or a color which is distinct from the color which is indicative of the pH value.

159. The method of claim 158, wherein the visualization surface is a primary color.

160. The method of claim 159, wherein the visualization surface is red, yellow, or blue.

161. The method of claim 158, wherein the visualization surface is a secondary color.

162. The method of claim 161, wherein the visualization surface is orange, green, or violet.

163. The method of claim 158, wherein the visualization surface is a tertiary color.

164. The method of claim 163, wherein the visualization surface is red-orange, yellow-orange, yellow-green, blue-green, blue-violet, or red-violet.

165. The method of any one of claims 84-164, wherein the method is suitable for detecting pH in saliva.

166. The method of any one of claims 84-165, wherein the method is suitable for detecting pH in gingival crevicular fluid.

167. A method of detecting a response to a remineralization treatment of a dental caries, and/or cavity or cavities, in a subject, comprising:
(a) contacting a surface of a tooth with the device of any one of claims 1-83, or with the sensing agent of any one of claims of any one of claims 1-32, 34-47, or 50-83, or a support and/or substrate comprising the sensing agent of any one of claims of any one of claims 1-32, 34-47, or 50-83; and
(b) determining a color change in the sensing agent, the color change being indicative of a pH value that is associated with the presence or absence of tooth demineralization, dental caries, and/or cavity or cavities, and/or risk of developing dental caries, and/or cavity or cavities, wherein the lack of a pH value associated with the presence of a dental caries, and/or cavity or cavities, indicative of a therapeutic response to the remineralization treatment.

168. A method of detecting a response to a remineralization treatment of a dental caries, and/or cavity or cavities, in a subject, comprising:
(a) contacting a surface of a tooth of the subject's with a sensing agent, or a support and/or substrate comprising the sensing agent, the sensing agent being configured to colorimetrically detect pH of saliva or gingival crevicular fluid; and
(b) determining a color change in the sensing agent, the color change being indicative of a pH value that is associated with the presence or absence of a dental caries, and/or cavity or cavities, caries risk, caries activity level, and/or risk of developing dental caries, and/or cavity or cavities,
wherein the lack of a pH value associated with the presence of a dental caries, and/or cavity or cavities, is indicative of a therapeutic response to a remineralization treatment.

169. The method of claim 167, wherein the remineralization treatment is selected from a fluoride agent, a calcium agent, and/or a phosphate agent.

170. The method of claim 169, wherein the fluoride agent is one or more of sodium fluoride, stannous fluoride, and acidulated phosphate fluoride.

171. The method of any one of claims 167-170, wherein the method prevents the need for a filling, crown, root canal, or extraction, optionally wherein the sensing agent detects the presence or absence of a dental caries, and/or cavity or cavities, for all surfaces of the teeth of the subject.

172. A method of preparing an anthocyanin, a carotene, or a curcumin from a food and/or spice source, comprising (a) obtaining the food and/or spice source and (b) lyophilizing the food and/or spice source or an extract from the food and/or spice source into a powder.

173. The method of claim 172, further comprising: (c) optionally dissolving the powder, or a medium containing the powder; (d) optionally vacuum filtering the powder to remove any fibrous or insoluble material associated with the food and/or spice source or extract; (e) optionally lyophilizing the filtrate; (f) optionally removing the sugars from the food and/or spice source; (g) optionally repeating step (e) once; and (h) optionally freeze drying the powder to produce the sensing agent concentrate.

174. The method of claim 172 or claim 173, wherein the food and/or spice source is a fruit or vegetable.

175. The method of any one of claims 172-174, wherein the powder is dissolved in water at about a 20% weight for weight proportion.

176. The method of any one of claims 172-175, wherein the lyophilizing is at about a pressure of 0.2 millibars.

177. The method of any one of claims 172-176, wherein the sugars from the food and/or spice source are removed by dissolving the filtrate in methanol, ethanol, sucrose, water mixtures, or water, or wherein the sugars from the food and/or spice source are removed by dissolving the filtrate in a sugar, a carbohydrate, or a PEG-based material.

178. The method of claim 177, wherein the sugars are dissolved in methanol at about a 20% weight for weight proportion.

179. The method of any one of claims 172-178, wherein the prepared anthocyanin concentrate is dissolved in water at about a 50% weight for weight proportion.

180. The method of any one of claims 172-179, wherein the prepared anthocyanin concentrate is for dip coating.
